(19) United States
(12) Patent Application Publication
(10) Pub. No.: US 2008/0085508 A1
(43) Pub. Date: Apr. 10, 2008

(54) Non-nucleic acid based biobarcode assay for detection of biological materials

(75) Inventors: Tai-Fen Wei, Vernon Hills, IL (US); Hao An, Arlington Heights, IL (US); Yijia Paul Bao, Vernon Hills, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Nanosphere, Inc., Northbrook, IL (US)

(21) Appl. No.: 11/803,012

(22) Filed: May 11, 2007

Related U.S. Application Data
(60) Provisional application No. 60/799,539, filed on May 11, 2006.

(51) Int. Cl. C12Q 1/68; C12Q 1/70; G01N 33/53; G01N 33/573

(52) U.S. Cl. 435/5; 435/6; 435/7.1; 435/7.4; 435/7.5

(57) ABSTRACT

The present invention relates to screening methods, compositions, and kits for detecting for the presence or absence of one or more target analytes, e.g. biomolecules, in a sample. In particular, the present invention relates to a method that utilizes non-nucleic acid reporter markers as biochemical barcodes for detecting multiple protein structures or other target analytes in a solution.

### CROSS-REFERENCE

This application claims the benefit of U.S. provisional patent application 60/799,539, filed May 11, 2006, which is incorporated by reference in its entirety.

### FIELD OF THE INVENTION

The present invention relates to a screening method for detecting for the presence or absence of one or more target analytes, e.g., proteins, nucleic acids, or other compounds in a sample. In particular, the present invention relates to a method that utilizes non-nucleic acid reporter markers as biochemical barcodes for detecting one or more analytes in a solution.

### BACKGROUND OF THE INVENTION

Every biological entity (e.g. viruses, bacteria, human cells) carries with it signature chemicals such as proteins and nucleic acid sequences that can serve as specific targets for detection. Methods for the detection of such diverse targets face many limitations due to the inadequate level of technological options presently available.

The detection of analytes is important for both molecular biology research and medical applications. Diagnostic methods based on fluorescence, mass spectroscopy, gel electrophoresis, laser scanning and electrochemistry are now available for identifying a variety of protein structures.¹⁻⁴ Antibody-based reactions are widely used to identify the genetic protein variants of blood cells, diagnose diseases, localize molecular probes in tissue, and purify molecules or effect separation processes.⁵ For medical diagnostic applications (e.g. malaria and HIV), antibody tests such as the enzyme-linked immunosorbent assay, Western blotting, and indirect fluorescent antibody tests are extremely useful for identifying single target protein structures.^{6,7}

Polymerase chain reaction (PCR) and other forms of target amplification have enabled rapid advances in the development of powerful tools for detecting and quantifying DNA targets of interest for research, forensic, and clinical applications.²⁶⁻³² The development of comparable target amplification methods for proteins could dramatically improve medical diagnostics and the developing field of proteomics.³³⁻³⁶ Although one cannot yet chemically duplicate protein targets, it is possible to tag such targets with oligonucleotide markers that can be subsequently amplified with PCR and then use DNA detection to identify the target of interest.³⁷⁻⁴⁵ This approach, often referred to as immuno-PCR, allows one to detect proteins with DNA labels in a variety of different formats. To date, all immuno-PCR approaches involve heterogeneous assays, which involve initial immobilization of a target analyte to a surface with subsequent detection using an antibody with a DNA label (for example, see U.S. Pat. Nos. 5,635,602, and 5,665,539). The DNA label is typically strongly bound to the antibody (either through covalent interactions or streptavidin-biotin binding).

For DNA detection methods, many assays have been developed using radioactive labels, molecular fluorophores, chemiluminescence schemes, electrochemical tags, and most recently, nanostructure-based labels.⁶¹⁻⁷⁰ Although some nanostructure-based methods are approaching PCR in terms of sensitivity, none thus far have achieved the 1-10 copy sensitivity level offered by PCR. Methods of synthesizing unique nanoparticle-oligonucleotide conjugates are well known, for example, in U.S. Pat. Nos. 6,750,016 and 6,506,564, which are hereby incorporated in their entirety. Previously, a method has been disclosed that utilizes reporter oligonucleotides as biochemical barcodes for detecting one or more analytes in a solution, as described in U.S. patent application Ser. No. 11/127,808, which is hereby incorporated in its entirety.

In the detection of specific nucleic acid molecules, the gold standards in sequence-specific detection are the polymerase chain reaction (PCR) and molecular fluorophore probe technology. PCR is an extraordinarily powerful technique. For protein targets, the enzyme-linked immunosorbent assay (ELISA) is the standard detection technique. The ELISA is an extremely general technique which relies on target-specific antibody labeling and calorimetric readout based either on fluorophores or chromophores. An alternative to these chemical detection assays that has recently been reported is the Biobarcode assay as disclosed in U.S. Ser. No. 10/877,750, filed Jun. 25, 2004 and U.S. Ser. No. 11/127,808, filed May 12, 2005, which are incorporated by reference in their entirety. This is a nanoparticle-based approach to the detection of protein and DNA targets (Nam J M, Thaxton C S, Mirkin C A Nanoparticle-based bio-bar codes for the ultrasensitive detection of proteins, Science 301 (5641):1884-1886 Sep. 26, 2003; Nam J M, Stoeva S I, Mirkin C A Bio-bar-code-based DNA detection with PCR-like sensitivity, J. Am. Chem. Soc. 126 (19):5932-5933 May 19, 2004.) The biobarcode assay takes advantage of two target-seeking probes.

### SUMMARY OF THE INVENTION

The present invention provides compositions and methods that greatly expand the flexibility, adaptability, multiplexing, and usefulness of techniques directed to the amplification of a signal to facilitate detection of a target analyte. The present invention also provides rapid and simultaneous sample screening for the presence of multiple antibodies, as well as easy, inexpensive, and time-saving simultaneous detection of several protein structures under assay conditions. For example, the present invention avoids the limited sensitivity problems due to low ratio of DNA identification sequence to detection antibody; slow target binding kinetics due to the heterogeneous nature of the target capture procedure, which increases assay time and decreases assay sensitivity; complex conjugation chemistries that are required to chemically link the antibody and DNA-markers; and a PCR amplification step. The present invention also provides methods and compositions that allow very high assay sensitivities, for example, proteins can be detected at pM ranges generally, without the need for expensive instruments. Thus, the present invention provides methods and compositions useful for detection of any target analyte. The methods and compositions of the invention can be used for point-of-care, research and clinical applications as well as for detection of environmental pollutants, toxins and biowarfare agents.

The present invention relates to methods, probes, compositions, and kits that utilize non-nucleic acid markers or reporters as biochemical barcodes for detecting at least one specific target analyte in one solution. The invention takes advantage of recognition elements of specific binding pairs functionalized either directly or indirectly with nanoparticles. For the detection of a target analyte, each recognition element of a specific binding pair can be associated with a different non-nucleic acid marker or reporter. The presence of a specific non-nucleic acid marker is indicative of the presence of the particular target analyte.

In a first aspect, the invention provides a nanoparticle probe for detecting for the presence of a target analyte in a sample, wherein the target analyte has at least two binding sites, the probe comprising a nanoparticle having bound thereto:

(i) a first member of a first specific binding pair; and

(ii) a capture probe comprising a specific binding complement of the target analyte,

wherein the first member of a first specific binding pair binds to a reporter, wherein the reporter comprises a non-nucleic acid linker, and wherein a second member of a first specific binding pair is bound to a first end of the linker.

In one embodiment of the first aspect, the capture probe further comprises a second member of the first specific binding pair and the reporter further comprises a first member of a second specific bind pair bound to a second end of the linker, wherein the reporter and the capture probe are bound to the first member of the first specific binding pair.

In another embodiment of the first aspect, the capture probe further comprises a second member of the first specific binding pair and the reporter further comprises a second member of a first specific bind pair bound to a second end of the linker, wherein the reporter and the capture probe are bound to the first member of the first specific binding pair.

In still another embodiment of the first aspect, the capture probe further comprises a second member of the first specific binding pair and a second nanoparticle having bound thereto the reporter, wherein a second end of the linker is bound to the second nanoparticle.

In yet another embodiment of the first aspect, the capture probe is bound to the nanoparticle and labeled with the first member of a first specific binding pair.

In second aspect, the invention provides a method for detecting for the presence or absence of a target analyte in a sample, wherein the target analyte has at least two binding sites the method comprising:

(a) providing a substrate and a nanoparticle probe according to any one of aspect I and embodiments Ia-Id

(b) immobilizing the target analyte onto the substrate;

(c) contacting the immobilized target analyte with the nanoparticle probe under conditions effective to allow for binding between the target analyte and the nanoparticle probe and the reporter to form a complex on the substrate;

(d) washing the substrate to remove unbound nanoparticle probes; and

(e) detecting for the presence or absence of the reporter, wherein the presence or absence of the reporter is indicative of the presence or absence of the target analyte in the sample.

In one aspect A, the invention provides a nanoparticle probe for detecting for the presence of a target analyte in a sample, wherein the target analyte has at least two binding sites, the probe comprising a nanoparticle having bound thereto:
(i) a first member of a first specific binding pair;
(ii) a capture probe comprising a specific binding complement of the target analyte labeled with a second member of the first specific binding pair; and
(iii) a reporter comprising a non-nucleic acid linker having two ends, a second member of a first specific binding pair bound to the first end of the linker and a first member of a second specific binding pair bound to the second end of the linker, wherein the reporter and capture probe are bound to the first member of the first specific binding pair.

In one embodiment of aspect A, the first specific binding pair comprises DNP/anti-DNP antibody or DIG/anti-DIG antibody.

In another embodiment of aspect A, the non-nucleic acid linkers comprise a polymer, 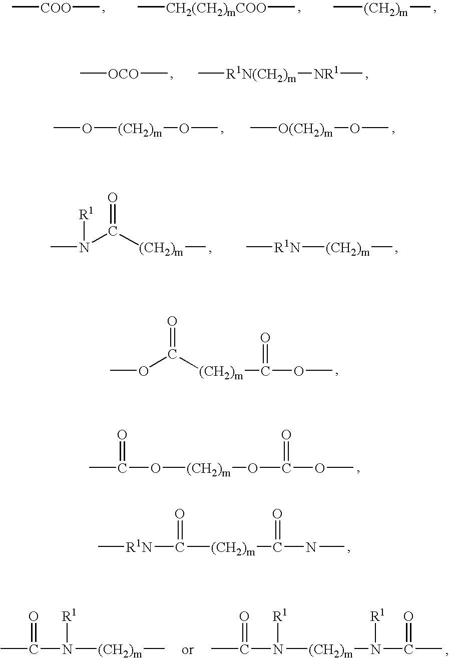
wherein:
R¹ has the formula X (CH₂)ₘ;
X is —CH₃, —CHCH₃, —COOH, —CO₂(CH₂)ₘCH₃, —OH, —CH₂OH, ethylene glycol, hexa(ethylene glycol), —O(CH₂)ₘCH₃, —NH₂, —NH(CH₂)ₘNH₂, halogen, glucose, maltose, fullerene C60, or a cyclic olefin; and
m is 0-30.

In another embodiment of aspect A, the second specific binding pair is biotin/streptavidin or biotin/avidin.

In another embodiment of aspect A, the nanoparticles are metal nanoparticles or semiconductor nanoparticles.

In another embodiment of aspect A, the nanoparticles are gold nanoparticles.

In one other embodiment of aspect A, the first and second specific binding pairs are independently an antibody and an antigen, a receptor and a ligand, an enzyme and a substrate, a drug and a target molecule, or the like.

In another embodiment of aspect A, the target has more than two binding sites.

In another embodiment of aspect A, at least two types of probes are provided, the first type of probe having a specific binding complement to a first binding site on the target analyte and the second type of probe having a specific binding complement to a second binding site on the target analyte. In another embodiment, a plurality of types of probes are provided, each type of probe having a specific binding complement to different binding sites on the target analyte.

In another embodiment of aspect A, the specific binding complement and the target analyte are members of a specific binding pair.

In still another embodiment of aspect A, members of the specific binding pair comprise nucleic acid, oligonucleotide, peptide nucleic acid, polypeptide, antibody, antigen, carbohydrate, protein, peptide, amino acid, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, or metabolites of or antibodies to any of the above substances. The nucleic acid and oligonucleotide comprise genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, or natural and synthetic nucleic acids.

In another embodiment of aspect A, wherein the target analyte is a nucleic acid and the specific binding complement is an oligonucleotide. In another embodiment, the target analyte is a protein or hapten and the specific binding complement is an antibody comprising a monoclonal or polyclonal antibody. In another embodiment, the target analyte is a sequence from a genomic DNA sample and the specific binding complements are oligonucleotides, the oligonucleotides having a sequence that is complementary to at least a portion of the genomic sequence. The genomic DNA is eukaryotic, bacterial, fungal or viral DNA.

In another embodiment of aspect A, the specific binding complement and the target analyte are members of an antibody-ligand pair.

In yet another embodiment of aspect A, in addition to its first binding site, the target analyte has been modified to include a second binding site.

In one other aspect B, the invention provides a method for detecting for the presence or absence of a target analyte in a sample, wherein the target analyte has at least two binding sites the method comprising:

(a) providing a first substrate and a nanoparticle probe comprising a nanoparticle having bound thereto: (i) a first member of a first specific binding pair;

(ii) a capture probe comprising a specific binding complement of the target analyte labeled with a second member of the first specific binding pair; and (iii) a reporter comprising a non-nucleic acid linker having two ends, a second member of a first specific binding pair bound to the first end of the linker and a first member of a second specific binding pair bound to the second end of the linker, wherein the reporter and capture probe are bound to the first member of the first specific binding pair;

(b) immobilizing the target analyte onto the first substrate;

(c) contacting the immobilized target analyte with the nanoparticle probe under conditions effective to allow for binding between the target analyte and the nanoparticle probe to form a complex on the substrate;

(d) washing the substrate to remove unbound nanoparticle probes; and

(e) detecting for the presence or absence of the reporter, wherein the presence or absence of the reporter is indicative of the presence or absence of the target analyte in the sample.

In one embodiment of aspect B, subsequent to step (d) and prior to step (e), the method further comprises step (d1) subjecting the complex to conditions effective to release the reporter. In another embodiment, prior to step (e), further comprising steps (d2) capturing the reporter onto a second substrate; (d2) contacting the immobilized reporter with a second nanoparticle probe, the second nanoparticle probe having a specific binding complement to the reporter, under conditions effective to allow binding between the reporter and the second nanoparticle probe to form a complex on the second substrate; and (d3) washing the second substrate to remove any unbound second nanoparticle probe. In another embodiment, step (e) detecting comprises contacting the washed second substrate with a stain. In another embodiment, the second substrate is a wave guide and step (e) comprises illuminating the substrate subsequent to step (d3) and observing for any changes in the intensity of light scattered.

In another embodiment of aspect B, the nanoparticles are metal nanoparticles or semiconductor nanoparticles. In another embodiment, the second nanoparticle probe is a gold nanoparticle probe.

In one other embodiment of aspect B, the specific binding pair is an antibody and an antigen, a receptor and a ligand, an enzyme and a substrate, a drug and a target molecule, or two strands of at least partially complementary oligonucleotides.

In another embodiment of aspect B, the target has more than two binding sites.

In still another embodiment of aspect B, at least two types of probes are provided, the first type of probe having a specific binding complement to a first binding site on the target analyte and the second type of probe having a specific binding complement to a second binding site on the target analyte. In another embodiment, a plurality of types of probes are provided, each type of probe having a specific binding complement to different binding sites on the target analyte.

In another embodiment of aspect B, the specific binding complement and the target analyte are members of a specific binding pair.

In yet another embodiment of aspect B, members of a specific binding pair comprise nucleic acid, oligonucleotide, peptide nucleic acid, polypeptide, antibody, antigen, carbohydrate, protein, peptide, amino acid, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, or metabolites of or antibodies to any of the above substances. In another embodiment, nucleic acid and oligonucleotide comprise genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, or natural and synthetic nucleic acids.

In another embodiment of aspect B, the target analyte is a nucleic acid and the specific binding complement is an oligonucleotide.

In another embodiment of aspect B, the target analyte is a protein or hapten and the specific binding complement is an antibody comprising a monoclonal or polyclonal antibody. In another embodiment, the target analyte is a sequence from a genomic DNA sample and the specific binding complements are oligonucleotides, the oligonucleotides having a sequence that is complementary to at least a portion of the genomic sequence. The genomic DNA is eukaryotic, bacterial, fungal or viral DNA.

In another embodiment of aspect B, the specific binding complement and the target analyte are members of an antibody-ligand pair.

In another embodiment of aspect B, in addition to its first binding site, the target analyte has been modified to include a second binding site.

In another aspect C, the invention provides a nanoparticle probe for detecting for the presence of a target analyte, wherein the target analyte is a first member of a first specific binding pair and wherein the target analyte has at least two binding sites, the probe comprising a nanoparticle having bound thereto:

(i) a first member of a second specific binding pair;

(ii) a capture probe comprising a second member of the first specific binding pair labeled with a second member of the second specific binding pair;

(iii) a reporter comprising a non-nucleic acid linker having two ends, a second member of a second specific binding pair bound to the first and second ends of the linker, wherein the reporter and capture probe are bound to the first member of the second specific binding pair.

In one embodiment of aspect C, the second specific binding pair is biotin/streptavidin or biotin/avidin. In another embodiment, the second member of the second specific binding pair is biotin.

In another aspect D, the invention provides a method for detecting for the presence or absence of a target analyte in a sample, wherein the target analyte has at least two binding sites, the method comprising:

(a) providing a first substrate and a nanoparticle probe comprising a nanoparticle having bound thereto: (i) a first member of a second specific binding pair; (ii) a capture probe comprising a second member of the first specific binding pair labeled with a second member of the second specific binding pair; (iii) a reporter comprising a non-nucleic acid linker having two ends, a second member of a second specific binding pair bound to the first and second ends of the linker, wherein the reporter and capture probe are bound to the first member of the second specific binding pair;

(b) immobilizing the target analyte onto the first substrate;

(c) contacting the immobilized target analyte with the probe under conditions effective to allow for binding interactions between the target analyte and the nanoparticle probe to form a complex on the substrate in the presence of the target analyte;

(d) washing the substrate to remove unbound nanoparticle probes; and

(e) detecting for the presence or absence of the reporter, wherein the presence or absence of the reporter is indicative of the presence or absence of the target analyte in the sample.

In one embodiment of aspect D, subsequent to step (d) and prior to step (e), further comprising step (d1) subjecting the complex to conditions effective to release the reporter. In another embodiment, prior to step (e), further comprising steps (d2) capturing the reporter onto a second substrate; (d3) contacting the immobilized reporter with a second nanoparticle probe, the second nanoparticle probe having a specific binding complement to the reporter, under conditions effective to allow binding between the reporter and the second nanoparticle probe and form a complex on the second substrate; and (d4) washing the second substrate to remove any unbound second nanoparticle probe. In another embodiment, step (e) detecting comprises contacting the washed second substrate with a stain. In another embodiment, the second nanoparticle probe is a gold nanoparticle probe. In still another embodiment, the second substrate is a waveguide and step (e) comprises illuminating the substrate subsequent to step (d4) and observing for any changes in the intensity of light scattered.

In another aspect E, the invention provides a method for detecting for the presence or absence of a target analyte in a sample, wherein the target analyte has at least two binding sites, the method comprising:

(a) providing a first substrate;

(b) providing a first nanoparticle probe comprising a nanoparticle having (i) a first member of a first specific binding pair bound thereto and (ii) a releasable specific binding complement to the target analyte, the specific binding complement labeled with a second member of the first specific binding pair;

(c) immobilizing the target analyte onto the first substrate;

(d) contacting the immobilized target analyte with the nanoparticle probe under conditions effective to allow for binding interactions between the target analyte and the first nanoparticle probe to form a complex on the substrate in the presence of the target analyte;

(e) washing the substrate to remove unbound first nanoparticle probes;

(f) releasing the specific binding complement from the first nanoparticle probe to form a second nanoparticle probe having the first member of the first specific binding pair; and

(g) detecting for the presence or absence of the second nanoparticle probe, wherein the presence or absence of the second nanoparticle probe is indicative of the presence or absence of the target analyte in the sample.

In one embodiment of aspect E, subsequent to step (f) and prior to step (g), further comprising steps (f1) capturing the second nanoparticle probe onto a second substrate having a second member of the first specific binding pair under conditions effective to allow binding interactions between the second nanoparticle probe and the second member of the first specific binding pair to form a complex on the second substrate in the presence of the second nanoparticle probe; and (f2) washing the second substrate to remove any unbound second nanoparticle probe. In another embodiment, step (g) detecting comprises contacting the washed second substrate with a stain. In still another embodiment, the second nanoparticle probe is a gold nanoparticle probe. In yet another embodiment, the second substrate is a waveguide and step (g) comprises illuminating the substrate subsequent to step (f2) and observing for any changes in the intensity of light scattered.

In another aspect F, the invention provides a nanoparticle probe comprising:
(i) a first nanoparticle having a first member of a non-nucleic acid specific binding pair bound thereto; and
(ii) a second nanoparticle having a non-nucleic acid linker bound thereto, the linker having a first end and a second end, wherein the first end of the linker is bound to the second nanoparticle and the second end of the linker is bound to a second member of the first specific binding pair, wherein the first and second nanoparticles are bound to each other by specific binding pair interactions.

In one embodiment of aspect F, the specific binding pair comprises is biotin/streptavidin or biotin/avidin. In another embodiment, the second member of the specific binding pair is biotin.

In another aspect G, the invention provides a method for detecting for the presence or absence of a target analyte in a sample, wherein the target analyte has at least two binding sites, the method comprising:

(a) providing a first substrate;

(b) providing a first nanoparticle probe comprising a nanoparticle having (i) a first member of a first specific binding pair bound thereto and (ii) a releasable specific binding complement to the target analyte, the specific binding complement labeled with a second member of the first specific binding pair;

(c) immobilizing the target analyte onto the first substrate;

(d) contacting the immobilized target analyte with the nanoparticle probe under conditions effective to allow for binding between the target analyte and the first nanoparticle probe to form a complex on the substrate in the presence of the target analyte;

(e) washing the first substrate to remove unbound first nanoparticle probes;

(f) releasing the specific binding complement from the first nanoparticle probe to form a second nanoparticle probe having the first member of the first specific binding pair;

(g) contacting the second nanoparticle probe with one or more third nanoparticle probes to form an aggregate probe in the presence of the second nanoparticle probe, the third nanoparticle probes comprising a nanoparticle having a non-nucleic acid linker molecule bound thereto, wherein a first end of the linker is bound to the third nanoparticle and a second end of the linker is bound to a second member of the first specific binding pair; and

(h) detecting for the presence or absence of the third nanoparticle probe, wherein the presence or absence of the third nanoparticle probe is indicative of the presence or absence of the target analyte in the sample.

In one embodiment of aspect G, subsequent to step (g) but prior to step (h), further comprising step (g1) isolating the aggregate probe; (g2) releasing the third nanoparticle probe from the aggregate probe; (g3) capturing the third nanoparticle probe onto a second substrate having a first member of the first specific binding pair; and (g4) washing the second substrate to remove any unbound third nanoparticle probe. In another embodiment, step (h) detecting comprises contacting the washed second substrate with a stain. In another embodiment, the second nanoparticle probe is a gold nanoparticle probe. In yet another embodiment, the second substrate is a waveguide and further comprising subsequent to step (g4), step (g5) illuminating the substrate and observing for any changes in the intensity of light scattered.

In another aspect H, the invention provides a nanoparticle probe for detecting for the presence of a target analyte, wherein the target analyte is a first member of a first specific binding pair, the probe comprising a nanoparticle having bound thereto a second member of the first specific binding pair, the second member of the first specific binding pair labeled with a first member of a second specific binding pair. In one embodiment, the second specific binding pair comprises is biotin/streptavidin or biotin/avidin. In another embodiment, the second member of the first specific binding pair is a target specific antibody and the first member of the second specific binding pair is biotin.

In another aspect I, the invention provides a method for detecting for the presence or absence of a target analyte in a sample, wherein the target analyte is a first member of a first specific binding pair, the method comprising:

(a) providing a first substrate;

(b) providing a first nanoparticle probe comprising a nanoparticle having bound thereto a second member of the first specific binding pair, the second member of the first specific binding pair labeled with a first member of a second specific binding pair;

(c) immobilizing the target analyte onto the first substrate;

(d) contacting the immobilized target analyte with the first nanoparticle probe under conditions effective to allow for binding interactions between the target analyte and the first nanoparticle probe to form a complex on the substrate in the presence of the target analyte;

(e) washing the substrate to remove unbound first nanoparticle probes;

(f) releasing the first nanoparticle probe; and

(g) detecting for the presence or absence of the first nanoparticle probe, wherein the presence or absence of the first nanoparticle probe is indicative of the presence or absence of the target analyte in the sample.

In one embodiment of aspect I, subsequent to step (f) and prior to step (g), further comprising steps (f1) capturing the first nanoparticle probe onto a second substrate having a second member of the first specific binding pair under conditions effective to allow binding interactions between the first nanoparticle probe and the second member of the first specific binding pair to form a complex on the second substrate in the presence of the second nanoparticle probe; and (f2) washing the second substrate to remove any unbound second nanoparticle probe. In another embodiment, step (g) detecting comprises contacting the washed second substrate with a stain. In one other embodiment, the second nanoparticle probe is a gold nanoparticle probe. In still another embodiment, the second substrate is a waveguide and step (g) comprises illuminating the substrate subsequent to step (f2) and observing for any changes in the intensity of light scattered.

In another aspect J, the invention provides a nanoparticle probe for detecting for the presence of a target analyte, the probe comprising a nanoparticle having bound thereto (i) a specific binding complement of a target analyte; and (ii) a first member of a first specific binding pair. In one embodiment, the first specific binding pair comprises is biotin/streptavidin or biotin/avidin. In another embodiment, the specific binding complement of the target analyte is a target specific antibody and the first member of the first specific binding pair is streptavidin.

In another aspect K, the invention provides a method for detecting for the presence or absence of a target analyte in a sample, wherein the target analyte has at least two binding sites, the method comprising:

(a) providing a first substrate;

(b) providing a first nanoparticle probe comprising a nanoparticle having bound thereto (i) a specific binding complement of a target analyte; and (ii) a first member of a first specific binding pair;

(c) immobilizing the target analyte onto the first substrate;

(d) contacting the immobilized target analyte with the first nanoparticle probe under conditions effective to allow for binding interactions between the target analyte and the first nanoparticle probe to form a complex on the substrate in the presence of the target analyte;

(e) washing the substrate to remove unbound first nanoparticle probes;

(f) releasing the first nanoparticle probe; and

(g) detecting for the presence or absence of the first nanoparticle probe, wherein the presence or absence of the first nanoparticle probe is indicative of the presence or absence of the target analyte in the sample.

In one embodiment of aspect K, subsequent to step (f) and prior to step (g), further comprising steps (f1) capturing the first nanoparticle probe onto a second substrate having a second member of the first specific binding pair under conditions effective to allow binding interactions between the first nanoparticle probe and the second member of the first specific binding pair to form a complex on the second substrate in the presence of the first nanoparticle probe; and (f2) washing the second substrate to remove any unbound first nanoparticle probe. In another embodiment, step (g) detecting comprises contacting the washed second substrate with a stain. In another embodiment, the second nanoparticle probe is a gold nanoparticle probe. In still another embodiment, the second substrate is a waveguide and step (g) comprises illuminating the substrate subsequent to step (f2) and observing for any changes in the intensity of light scattered.

In another aspect L, the invention provides a method for detecting for the presence or absence of a target analyte in a sample, wherein the target analyte has at least two binding sites, the method comprising:

(a) providing a first substrate and a second substrate;

(b) labeling a sample believed to have the target analyte with a first member of a first specific binding pair;

(c) immobilizing labeled target analyte onto the first substrate;

(d) washing the first substrate to remove unbound labeled target analyte;

(e) releasing the labeled target analyte;

(f) recapturing the labeled target analyte onto the second substrate;

(g) contacting the recaptured target analyte with the a nanoparticle probe comprising a nanoparticle having a second member of the first specific binding pair under conditions effective to allow for binding between the recaptured labeled target analyte and the first nanoparticle probe to form a complex on the substrate in the presence of the labeled target analyte;

(h) washing the substrate to remove unbound nanoparticle probes; and

(i) detecting for the presence or absence of the nanoparticle probe, wherein the presence or absence of the nanoparticle probe is indicative of the presence or absence of the target analyte in the sample.

In one embodiment of aspect L, step (i) detecting comprises contacting the washed second substrate with a stain.

In another embodiment of aspect L, the nanoparticle probe is a gold nanoparticle probe.

In another embodiment of aspect L, the second substrate is a waveguide and step (i) comprises illuminating the substrate subsequent to step (h) and observing for any changes in the intensity of light scattered.

In another aspect M, the invention provides a method for detecting for the presence or absence of a target analyte in a sample, wherein the target analyte has at least two binding sites, the method comprising:

(a) providing a first substrate and a second substrate;

(b) providing a first nanoparticle probe comprising a nanoparticle having (i) a first member of a first specific binding pair bound thereto and (ii) a specific binding complement to the target analyte, the specific binding complement labeled with a second member of the first specific binding pair;

(c) providing a second nanoparticle probe comprising a nanoparticle having a non-nucleic acid linker molecule bound thereto, wherein a first end of the linker is bound to the second nanoparticle and a second end of the linker is bound to a second member of the first specific binding pair;

(d) immobilizing the target analyte onto the first substrate;

(e) contacting the immobilized target analyte with the nanoparticle probe under conditions effective to allow for binding interactions between the target analyte and the first nanoparticle probe to form a complex on the substrate in the presence of the target analyte;

(f) washing the first substrate to remove unbound first nanoparticle probes;

(g) releasing the first member of the first specific binding pair from the first nanoparticle probe;

(h) immobilizing the first member of the first specific binding pair onto the second substrate;

(i) washing the second substrate to remove unbound first member of the specific binding pair;

(j) contacting the captured first member of the first specific binding pair on the second substrate with the second nanoparticle probe under conditions effective to allow for binding between the captured first member of the first specific binding pair and the second nanoparticle probe to form a complex in the presence of the first member;

(k) washing the second substrate so as to remove unbound second nanoparticle probes; and

(l) detecting for the presence or absence of the second nanoparticle probe, wherein the presence or absence of the second nanoparticle probe is indicative of the presence or absence of the target analyte in the sample.

In one embodiment of aspect M, step (l) detecting comprises contacting the washed second substrate with a stain.

In another embodiment of aspect M, the second nanoparticle probe is a gold nanoparticle probe.

In still another embodiment of aspect M, the second substrate is a waveguide and step (l) comprises illuminating the substrate and observing for any changes in the intensity of light scattered.

In another aspect N, the invention provides a method for detecting for the presence or absence of a target analyte in a sample, wherein the target analyte has at least two binding sites the method comprising:

(a) providing a first substrate;

(b) providing a first particle probe comprising a polyacrylic acid polymer having bound thereto a specific binding complement of the target analyte; and (ii) a first member of a first specific binding pair;

(c) immobilizing the target analyte onto the first substrate;

(d) contacting the immobilized target analyte with the first particle probe under conditions effective to allow for binding interactions between the target analyte and the first particle probe to form a complex on the first substrate in the presence of the target analyte;

(e) washing the first substrate to remove unbound first particle probes;

(f) denaturing the first particle probe to form fragments; and

(g) detecting for the presence or absence of the fragments, wherein the presence or absence of the fragments is indicative of the presence or absence of the target analyte in the sample.

In one embodiment of aspect N, subsequent to step (f) and prior to step (g), further comprising steps (f1) capturing the fragments onto a second substrate having a second member of the first specific binding pair under conditions effective to allow binding interactions between the fragments and the second member of the first specific binding pair and form a complex on the second substrate in the presence of the fragments; (f2) washing the second substrate to remove any unbound fragments; and (f3) contacting the fragments bound to the second substrate with a nanoparticle probe comprising a nanoparticle having bound thereto the second member of the first specific binding pair. In another embodiment, step (g) detecting comprises contacting the washed second substrate with a stain. In one other embodiment, the second nanoparticle probe is a gold nanoparticle probe. In still another embodiment, the second substrate is a waveguide and step (g) comprises illuminating the substrate subsequent to step (f3) and observing for any changes in the intensity of light scattered.

In another aspect O, the invention provides a method for detecting for the presence or absence of a target analyte in a sample, wherein the target analyte has at least two binding sites, the method comprising:

(a) providing a first substrate and a second substrate having a first member of a first specific binding pair bound thereto;

(b) providing a specific binding complement to the target analyte, the specific binding complement labeled with a second member of the first specific binding pair;

(c) providing a nanoparticle probe comprising a nanoparticle having a second member of the first specific binding pair bound thereto;

(d) immobilizing the target analyte onto the first substrate;

(e) contacting the immobilized target analyte with the specific binding complement under conditions effective to allow for binding between the target analyte and the specific binding complement to form a complex on the first substrate in the presence of the target analyte;

(f) washing the first substrate to remove unbound specific binding complement;

(g) releasing specific binding complement;

(h) capturing the released specific binding complement onto the second substrate;

(i) washing the second substrate to remove unbound specific binding complements;

(j) contacting the captured specific binding complement on the second substrate with the nanoparticle probe under conditions effective to allow for binding between the captured specific binding complement and the nanoparticle probe to form a complex in the presence of captured specific binding complement;

(k) washing the second substrate so as to remove unbound nanoparticle probe; and

(l) detecting for the presence or absence of the nanoparticle probe, wherein the presence or absence of the nanoparticle probe is indicative of the presence or absence of the target analyte in the sample.

In one embodiment of aspect O, step (l) detecting comprises contacting the washed second substrate with a stain. In another embodiment, the nanoparticle probe is a gold nanoparticle probe. In still another embodiment, the second substrate is a wave guide and step (l) comprises illuminating the substrate and observing for any changes in the intensity of light scattered.

In another aspect P, the invention provides a kit for detecting for one or more target analytes in a sample, the kit comprising the nanoparticle probe of any one of aspects A, C, F, H and J and an optional substrate.

### DESCRIPTION OF THE DRAWINGS

FIG. 1A describes a nanoparticle detection probe loaded with anti-DNP antibodies. The capture substrate shown is a magnetic bead, coated with a first capture moiety which is an antibody to the target analyte. The capture substrate, DNP-labeled second capture moieties, nanoparticle, and biotin-DNP labeled non-nucleic acid markers, and the target analyte form a complex. After separating the complexes from the sample solution, and washing unbound nanoparticle detection probes, the non-nucleic acid markers, here biotin-DNP non-nucleic acid markers, are released. The released non-nucleic acid markers are then bound to a substrate coated with anti-DNP antibodies, detected with nanoparticle probes after silver amplification. The presence of the marker indicates the presence of the target analyte in the sample.

FIG. 1B describes a nanoparticle detection probe loaded with anti-DIG antibodies. The capture substrate shown is a magnetic bead, coated with a first capture moiety which is an antibody to the target analyte. The capture substrate, DIG-labeled second capture moieties, nanoparticle, biotin-DIG labeled non-nucleic acid markers and the target analyte form a complex. After separating the complexes from the sample solution, and washing unbound nanoparticle detection probes, the non-nucleic acid markers, here biotin-DIG non-nucleic acid markers, are released. The released non-nucleic acid markers are then bound to a substrate coated with anti-DIG antibodies, detected with nanoparticle probes after silver amplification. The presence of the markers indicates the presence of the target analyte in the sample.

FIG. 2A describes a nanoparticle detection probe coated in streptavidin. The capture substrate shown is a magnetic bead, coated with a first capture moiety which is an antibody to the target analyte. The capture substrate, biotin labeled second anti-target antibody, nanoparticle, biotin-biotin non-nucleic acid markers and the target analyte form a complex. After separating the complexes from the sample solution, and washing unbound nanoparticle detection probes, the nanoparticle detection probe bound with biotin-biotin non-nucleic acid markers are released from the complex. The released nanoparticle detection probes are then bound to a substrate coated with streptavidin, detected with nanoparticle probes after silver amplification. The presence of the marker indicates the presence of the target analyte in the sample.

FIG. 2B describes a nanoparticle detection probe coated with streptavidin is loaded with biotin-biotin non-nucleic acid markers and biotinylated nucleic acid complementary to at least one portion of the target nucleic acid analyte. The capture substrate shown is a magnetic bead coated with nucleic acids complementary to at least one portion of the target nucleic acid analyte. After removing the complexes from the sample solution, and washing unbound nanoparticle detection probes, biotin-biotin non-nucleic acid markers are released from the complex. The released markers are then bound to a substrate coated with streptavidin, detected with nanoparticle probes after silver amplification. The presence of the markers indicate the presence of the target analyte in the sample.

FIG. 3 describes a nanoparticle detection probe coated with streptavidin and biotin-labeled target-specific second capture moieties, in this case, antibodies, bound thereto. The capture substrate shown is a magnetic bead coated with a first kind of target-specific capture moiety, in this case, antibodies. After separating the complexes from the sample solution, and washing away unbound nanoparticle detection probes, the bound nanoparticle detection probes coated with streptavidin are released from the complex. The released nanoparticle detection probes coated with streptavidin are then bound to a substrate coated with biotinylated captures, and detected by silver enhancement. The presence of the nanoparticle detection probe coated with streptavidin indicates the presence of the target analyte in the sample.

FIG. 4 describes a nanoparticle probe coated with streptavidin and biotin-labeled target-specific second capture moieties, in this case, antibodies, bound thereto. The capture substrate shown is a magnetic bead coated with a first kind of target-specific capture moiety, in this case, antibodies. After separating the complexes from the sample solution, and washing unbound nanoparticle probes, biotin-loaded detection nanoparticles are added to bind the complex through the bound nanoparticle probe. Unbound biotin nanoparticles are washed away and the complex bound biotin nanoparticles are released and detected on a streptavidin-coated surface by silver enhancement.

FIG. 5 describes a nanoparticle detection probe coated with biotin-labeled target-specific second capture moieties, in this case, antibodies, bound thereto. The capture substrate shown is a magnetic bead coated with a first kind of target-specific capture moiety, in this case, antibodies. After separating the complexes from the sample solution, and washing unbound nanoparticle detection probes, the nanoparticle detection probes are released from the complex. The released nanoparticle detection probes are then bound to a substrate coated with streptavidin captures, and detected directly by silver enhancement. The presence of the nanoparticle detection probe indicates the presence of the target analyte in the sample.

FIG. 6 describes nanoparticle detection probes co-loaded with streptavidin and a second capture moiety, in this case, target-specific antibodies. The capture substrate shown is a magnetic bead coated with a first target-specific capture moiety, in this case, antibodies. After separating the complexes from the sample solution, and washing unbound nanoparticle detection probes, the nanoparticle detection probes are released from the complex. The released nanoparticle detection probes are then bound to a substrate coated with biotinylated captures, and detected directly by silver enhancement. The presence of the nanoparticle detection probe indicates the presence of the target analyte in the sample.

FIG. 7 describes a method comprising capture substrates that are magnetic beads coated with a first capture moiety, in this case, target analyte-specific antibodies. The target analytes are biotinylated. After the first capture moiety binds the target analyte specifically, the complex may be separated from solution and washed to remove unbound moieties in the sample. The biotinylated target is released from the magnetic bead, and captured on a streptavidin array. Streptavidin-coated signal probes (nanoparticles) are added to generate signal.

FIG. 8 describes nanoparticle detection probes loaded with streptavidin, and bound with a second capture moiety, in this case, biotin-labeled target-specific antibodies. The capture substrate shown is a magnetic bead coated with a first target-specific capture moiety, in this case, antibodies. After separating the complexes from the sample solution, and washing unbound nanoparticle detection probes, the streptavidin is released from the nanoparticle detection probes. The released streptavidin are then bound to a substrate coated with biotinylated captures. Biotin-loaded nanoparticles are then added as signal probe which are detected by silver enhancement. The presence of released streptavidin indicates the presence of the target analyte in the sample.

FIG. 9 describes a method comprising capture substrates that are magnetic beads coated with a first capture moiety, in this case, target analyte-specific antibodies. The detection probe comprises biotin-conjugated polyacrylic acid polymers. Streptavidin is added to bind the biotinylated polymer and the biotin-labeled second capture moiety. After the first capture moiety binds the target analyte, then complexed with free streptavidin, and biotinylated polymer, the complex may be separated from solution and washed to remove unbound biotin-conjugated polyacrylic acid polymers. The polymers are then released and fragmented to expose the biotin molecules. The polymers with exposed biotin labels are captured on a streptavidin array. Streptavidin-coated nanoparticle probes are added for signal detection by silver enhancement.

FIG. 10 describes a method comprising capture substrates that are magnetic beads coated with a first capture moiety, in this case, target analyte-specific antibodies. Biotin-labeled second capture moieties are added to target analytes bound to the first capture moiety. Once the complex is formed, unbound biotinylated capture moieties can be removed by washing. The biotinylated second capture moieties are then released, and captured on a streptavidin array. Streptavidin-coated nanoparticles are added for signal detection by silver enhancement.

FIG. 11 describes Prostate Specific Antigen (PSA) target detection used as an example to illustrate, but not to limit, the invention. PSA target was tested from 100 pg, 10 pg, 1 pg, 100 fg, 10 fg, 1 fg to 0 fg per assay. Different amounts of target was first captured using 2 μg magnetic beads (MB) [Dynabeads® Myone™ Tosylactivated, coated with PSA antibody [Biodesign, MAb, A-PSA free form, Cat#M86806M, Lot #21k31504, clone #8A6] in 200 uL of Barcode Buffer (1×PBS [Gibco, Cat #70013-032, Lot#1148371] 0.5% BSA [R&D System, Cat#Dy995, part#841380, Lot#225340], 0.05% Tween 20 [SigmaUltra, P-7949, Lot#81K0293]) at 25° C. with shaking at 200 rpm for 90 minutes. To form a specific sandwich, 100 ng of the biotinylated anti-human Kallikrein 3 polyclonal goat IgG [anti-PSA-biotin AB, R&D System cat#BAF1344, Lot#IR013071] is added as a secondary antibody and incubated for an additional one hour at 25° C. with shaking at 1200 rpm. After two times washing with Barcode Buffer, 1 μL of the streptavidin coated nanoparticles was added. The bound streptavidin coated nanoparticles (a component of the specific complex) are released and applied to a biotin printed microarray. Array binding reaction was performed in 50 μL buffer (1×PBS, 0.025% Tween 20, 0.05% BSA) incubated at 25° C. with shaking at 800 rpm for 1 hour. After washing with 0.5N NaNO₃ four times, array was developed with silver and signals measured with light scattering. The scanned image and data analysis were shown in FIG. 11.

### DESCRIPTION OF THE INVENTION

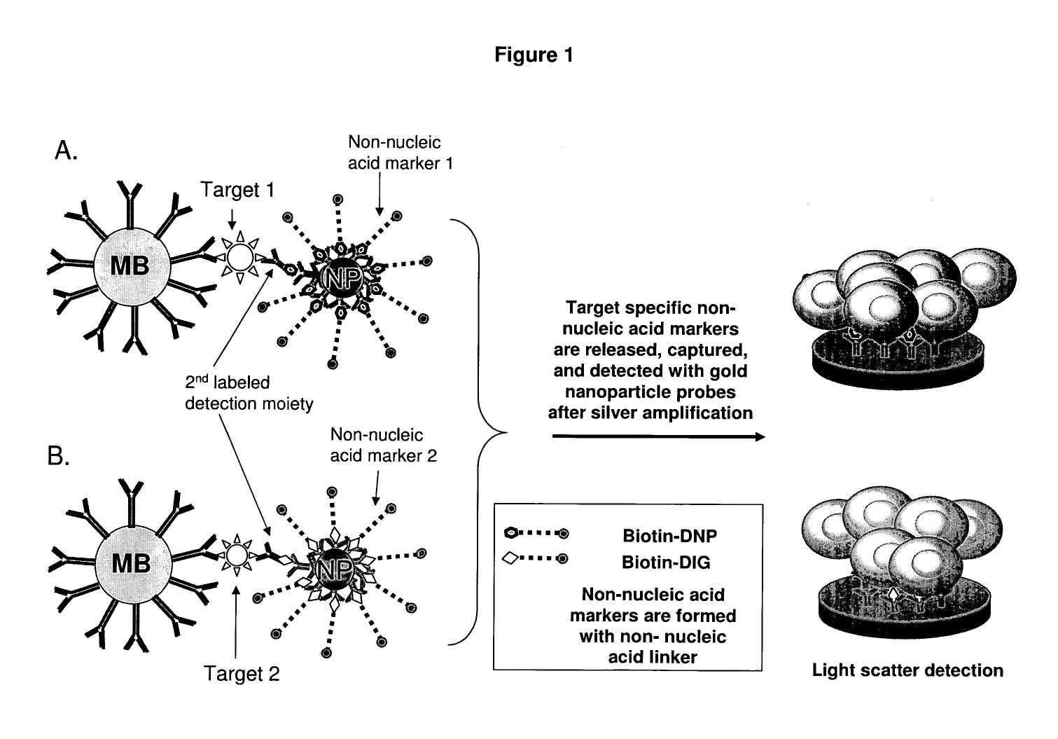

The current invention overcomes many of the problems of the prior art while greatly expanding the flexibility, adaptability and usefulness of techniques directed to the amplification of a signal to facilitate detection. FIG. 1-11 illustrate certain embodiments of the invention, but do not limit the invention in any way. The present invention relates to methods, probes, compositions, and kits that utilize non-nucleic acid receptor as biochemical barcodes for detecting at least one specific target analyte in one solution. The approach takes advantage of recognition elements of specific binding pairs functionalized either directly or indirectly with nanoparticles, and the previous observation that hybridization events that result in the aggregation of gold nanoparticles can significantly alter their physical properties (e.g. optical, electrical, mechanical).⁸⁻¹²

As used herein, a “type of” nanoparticles, conjugates, particles, latex microspheres, etc. having non-nucleic acid markers attached thereto refers to a plurality of that item having the same type(s) of non-nucleic acid markers attached to them. “Nanoparticles having non-nucleic acid markers attached thereto” are also sometimes referred to as “nanoparticle detection probes” or, in the case of the detection methods of the invention, “nanoparticle probes,” or just “probes.”

As used herein, the term “particle” refers to a small piece of matter that can preferably be composed of metals, silica, silicon-oxide, or polystyrene. A “particle” can be any shape, such as spherical or rod-shaped. The term “particle” as used herein specifically encompasses both nanoparticles and nanoparticles as defined and described hereinbelow.

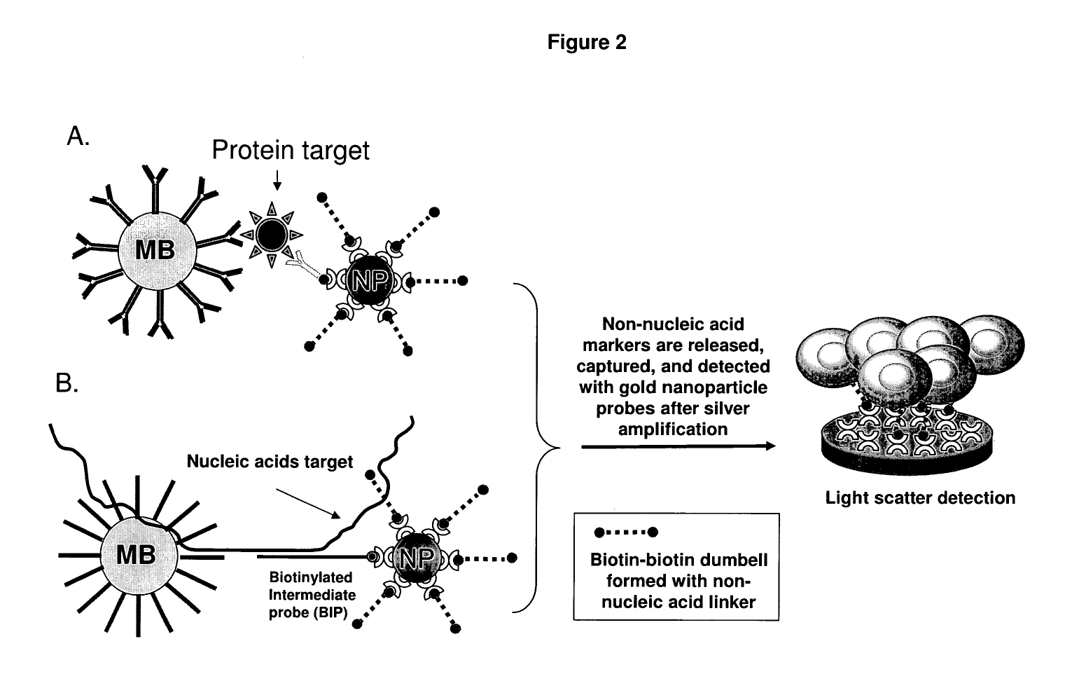

As used throughout the invention “non-nucleic acid marker”, “barcode”, “biochemical barcode”, “biobarcode”, “reporter barcode”, or “reporter”, etc. are all interchangeable with each other and have the same meaning. Preferably, the non-nucleic acid marker comprises two markers linked by a non-nucleic acid linker, represented by a “dumbbell” shape in FIGS. 1 and 2. The markers may be the same, or may be different. For instance, one marker could be biotin, and the other could be DNP (dinitrophenol). In other embodiments, the two markers are biotin and DIG (digoxigenin), or biotin and biotin. If desired, the non-nucleic acid marker may be labeled, for instance, with a radiolabel or a fluorescent label. Alternatively, the non-nucleic acid marker may comprise at least one member of a specific binding pair directly bound to the marker. For example, a member of a specific binding pair may be bound to each end of the marker.

As used throughout this invention, “non-nucleic acid marker receptor” and “non-nucleic acid receptor” are interchangeable, and refer to a receptor which coats the nanoparticle.

A “non-nucleic acid” refers to any molecule other than molecules that consist of nucleic acids, such as DNA and RNA. Accordingly, a “non-nucleic acid linker” refers to any molecule comprising a non-nucleic acid molecule. Such linkers can be, but not limited to, a polymer,

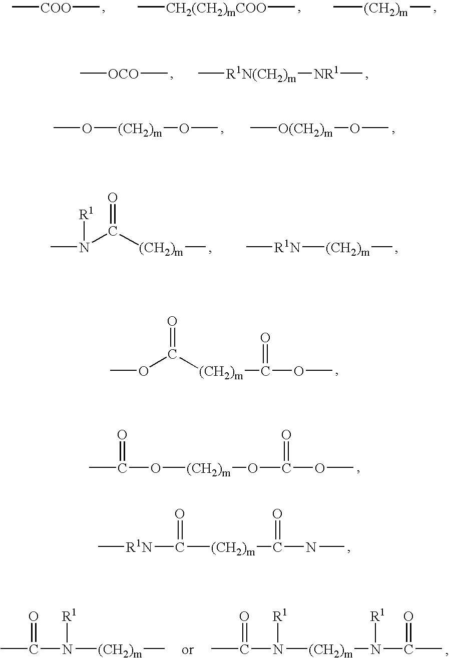

wherein:
R¹ has the formula X (CH₂)ₘ;
X is —CH₃, —CHCH₃, —COOH, —CO₂(CH₂)ₘCH₃, —OH, —CH₂OH, ethylene glycol, hexa(ethylene glycol), —O(CH₂)ₘCH₃, —NH₂, —NH(CH₂)ₘNH₂, halogen, glucose, maltose, fullerene C60, or a cyclic olefin; and
m is 0-30.

The term “analyte” or “target analyte” refers to the compound or composition to be detected, including, but not limited to, drugs, metabolites, pesticides, pollutants, proteins, peptides, nucleic acid segments, molecules, cells, microorganisms and fragments and products thereof, or any substance for which attachment sites, binding members or receptors (such as antibodies) can be developed, and the like. The analyte can be comprised of a member of a specific binding pair (sbp) and may be a ligand, which is monovalent (monoepitopic) or polyvalent (polyepitopic), preferably antigenic or haptenic, and is a single compound or plurality of compounds, which share at least one common epitopic or determinant site. The analyte can be a part of a cell such as bacteria or a cell bearing a blood group antigen such as A, B, D, O, etc., or an HLA antigen or a microorganism, e.g., bacterium, fungus, protozoan, or virus. If the analyte is monoepitopic, the analyte can be further modified, e.g. chemically, to provide one or more additional binding sites. In practicing this invention, the analyte has at least two binding sites, e.g., epitopes or binding sites that can be targeted by a capture probe, specific binding complement or a capture moiety.

The polyvalent ligand analytes will normally be larger organic compounds, often of polymeric nature, such as polypeptides and proteins, polysaccharides, nucleic acids, and combinations thereof. Such combinations include components of bacteria, viruses, chromosomes, genes, mitochondria, nuclei, cell membranes and the like.

For the most part, the polyepitopic ligand analytes to which the subject invention can be applied will have a molecular weight of at least about 5,000, more usually at least about 10,000. In the polymeric molecule category, the polymers of interest will generally be from about 5,000 to 5,000,000 molecular weight, more usually from about 20,000 to 1,000,000 molecular weight; among the hormones of interest, the molecular weights will usually range from about 5,000 to 60,000 molecular weight.

A wide variety of proteins may be considered as belonging to the family of proteins having similar structural features, proteins having particular biological functions, proteins related to specific microorganisms, particularly disease causing microorganisms, etc. Such proteins include, for example, immunoglobulins, cytokines, enzymes, hormones, cancer antigens, nutritional markers, tissue specific antigens, etc.

The types of proteins, blood clotting factors, protein hormones, antigenic polysaccharides, microorganisms and other pathogens of interest in the present invention are specifically disclosed in U.S. Pat. No. 4,650,770, the disclosure of which is incorporated by reference herein in its entirety.

The monoepitopic ligand analytes will generally be from about 100 to 2,000 molecular weight, more usually from 125 to 1,000 molecular weight.

The analyte may be a molecule found directly in a sample such as a body fluid from a host. The sample can be examined directly or may be pretreated to render the analyte more readily detectable. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like.

The term “specific binding pair (sbp) member” refers to one of two molecules, which specifically binds to and can be defined as complementary with a particular spatial and/or polar organization of the other molecule. The members of the specific binding pair can be referred to as ligand and receptor (antiligand). These will usually be members of an immunological pair such as antigen-antibody, although other specific binding pairs such as biotin-avidin, enzyme-substrate, enzyme-antagonist, enzyme-agonist, drug-target molecule, hormones-hormone receptors, nucleic acid duplexes, IgG-protein A/protein G, antibody-ligand, polynucleotide pairs such as DNA-DNA, DNA-RNA, protein-DNA, lipid-DNA, lipid-protein, polysaccharide-lipid, protein-polysaccharide, nucleic acid aptamers and associated target ligands (e.g., small organic compounds, nucleic acids, proteins, peptides, viruses, cells, etc.), and the like are not immunological pairs but are included in the invention and the definition of sbp member. A member of a specific binding pair can be the entire molecule, or only a portion of the molecule so long as the member specifically binds to the binding site on the target analyte to form a specific binding pair.

In the phrase “first member of a first specific bind pair” or “second member of a first specific bind pair” or the like, the “first member” and “second member” serve only to denote one member of the pair and to track the member of a specific binding pair involved in a binding interaction. For example, in the a specific bind pair X-Y, X can be the first member and Y the second member, or Y can be the first member and X the second member.

The term “ligand” refers to any organic compound for which a receptor naturally exists or can be prepared. The term ligand also includes ligand analogs, which are modified ligands, usually an organic radical or analyte analog, usually of a molecular weight greater than 100, which can compete with the analogous ligand for a receptor, the modification providing means to join the ligand analog to another molecule. The ligand analog will usually differ from the ligand by more than replacement of a hydrogen with a bond, which links the ligand analog to a hub or label, but need not. The ligand analog can bind to the receptor in a manner similar to the ligand. The analog could be, for example, an antibody directed against the idiotype of an antibody to the ligand.

The term “receptor” or “antiligand” refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, nucleic acid aptamers, avidin, protein A, barstar, complement component C1q, and the like. Avidin is intended to include egg white avidin and biotin binding proteins from other sources, such as streptavidin.

The term “specific binding” refers to the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide interactions, and so forth.

The term “non-specific binding” refers to the binding between molecules that is relatively independent of specific surface structures. Non-specific binding may result from several factors including hydrophobic interactions between molecules.

The term “antibody” refers to an immunoglobulin which specifically binds to and is thereby defined as complementary with a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained.

A specific binding complement may be a member of a specific binding pair. The following are non-limiting examples of target analyte:specific binding complements. A target analyte can be a nucleic acid and the specific binding complement can be an oligonucleotide. A target analyte can be a protein or hapten and the specific binding complement can be an antibody comprising a monoclonal or polyclonal antibody. A target analyte can be a sequence from a genomic DNA sample and the specific binding complements can be oligonucleotides, the oligonucleotides having a sequence that is complementary to at least a portion of the genomic sequence. Genomic DNA can be eukaryotic, bacterial, fungal or viral DNA. A target analyte can be a sequence from episomal DNA sample and the specific binding complements can be oligonucleotides, the oligonucleotides having a sequence that is complementary to at least a portion of the episomal DNA sequence. A specific binding complement and the target analyte can be members of an antibody-ligand pair.

The term “capture probe” and “second capture moiety” are used interchangeably and to refer to any compound, complex, molecule or entity, such as antibody, oligonucleotide, aptamer, lectin or similar material, that is capable of selectively and specifically binding to the target species of interest.

A “capture substrate”, “first capture moiety” or “substrate” can be any insoluble material to which analytes can be immobilized as described above and throughout this disclosure. A “capture substrate” as used herein has bound thereto a specific binding complement that binds to the target and captures the target analytes from a sample, and can facilitate the separation of these captured target analytes (both before and after treatment with the detection probe) from the sample. Such substrates are typically physically large relative to the analyte and are preferably insoluble in the sample. In particular instances, the methods of the invention comprise the use of magnetic substrates, as described herein, which can be isolated by subjecting the magnetic substrate to a magnetic field.

As used herein, the terms “label” or “detection label” refers to a detectable marker that may be detected by photonic, electronic, opto-electronic, magnetic, gravity, acoustic, enzymatic, or other physical or chemical means. The term “labeled” refers to incorporation of such a detectable marker, e.g., by incorporation of a radiolabeled nucleotide or attachment of a detectable marker. If desired, the non-nucleic acid markers may optionally include detection labels including, but are not limited to, fluorophores, chromophores, oligonucleotides with or without attached fluorophores or chromophores, proteins including enzymes and porphyrins, lipids, carbohydrates, synthetic polymers and tags such as isotopic or radioactive tags.

Polyclonal antibodies directed toward a target analyte generally are raised in animals (e.g., rabbits or mice) by multiple subcutaneous or intraperitoneal injections of JNK activating phosphatase polypeptide and an adjuvant. It may be useful to conjugate an target analyte protein, polypeptide, or a variant, fragment or derivative thereof to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for anti-target analyte antibody titer.

Monoclonal antibodies directed toward target analytes are produced using any method that provides for the production of antibody molecules by continuous cell lines in culture. Examples of suitable methods for preparing monoclonal antibodies include hybridoma methods of Kohler, et al., Nature 256:495-97 (1975), and the human B-cell hybridoma method, Kozbor, J. Immunol. 133:3001 (1984); Brodeur et al., Monoclonal Antibody Production Techniques and Applications 51-63 (Marcel Dekker 1987).

The term “oligonucleotide” referred to herein includes naturally occurring, and modified nucleotides linked together by naturally occurring, and/or non-naturally occurring oligonucleotide linkages. Oligonucleotides are a polynucleotide subset comprising members that are generally single-stranded and have a length of 200 bases or fewer. In certain embodiments, oligonucleotides are 10 to 60 bases in length. In certain embodiments, oligonucleotides are 10, 11, 12, 13, 14, 15, 16, 17, 18, 19, or 20 to 40 bases in length. Oligonucleotides may be single stranded or double stranded, e.g. for use in the construction of a gene mutant. Oligonucleotides of the invention may be sense or antisense oligonucleotides with reference to a protein-coding sequence.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

“Nanoparticles” useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, In₂Se₃, In₂Se₃, Cd₃P₂, Cd₃As₂, InAs, and GaAs. The size of the nanoparticles is preferably from about 5 nm to about 150 nm (mean diameter), more preferably from about 5 to about 50 nm, most preferably from about 10 to about 30 mm. The nanoparticles may also be rods. Other nanoparticles useful in the invention include silica and polymer (e.g. latex) nanoparticles.

Methods of making metal, semiconductor and magnetic nanoparticles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988), all of which are incorporated by reference in their entirety. Methods of making silica nanoparticles impregnated with fluorophores or phosphors are also well known in the art (see Tan and coworkers, PNAS, 2004, 101, 15027-15032, which is incorporated by reference in its entirety).

Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky et al., J. Am. Chem. Soc., 112, 9438 (1990); Ushida et al., J. Phys. Chem., 95, 5382 (1992), all of which are incorporated by reference in their entirety.

A “sample” as used herein refers to any quantity of a substance that comprises potential target analytes and that can be used in a method of the invention. For example, the sample can be a biological sample or can be extracted from a biological sample derived from humans, animals, plants, fungi, yeast, bacteria, viruses, tissue cultures or viral cultures, or a combination of the above. They may contain or be extracted from solid tissues (e.g. bone marrow, lymph nodes, brain, skin), body fluids (e.g. serum, blood, urine, sputum, seminal or lymph fluids), skeletal tissues, or individual cells. Alternatively, the sample can comprise purified or partially purified nucleic acid molecules or proteins and, for example, buffers and/or reagents that are used to generate appropriate conditions for successfully performing a method of the invention.

In one embodiment, metallic nanoparticles are employed as a light-scattering label in a method of the invention. Such labels cause incident light to be scattered elastically, i.e. substantially without absorbing light energy. Suitable but non-limiting nanoparticles and methods for preparing such nanoparticles are described in U.S. Pat. No. 6,506,564, issued Jan. 14, 2003; U.S. Ser. No. 10/854,848, filed May 27, 2004; U.S. Ser. No. 10/995,051, filed Nov. 22, 2004; U.S. Ser. No. 09/820,279, filed Mar. 28, 2001; U.S. Ser. No. 008,978, filed Dec. 7, 2001; U.S. Ser. No. 10/125,194, filed Apr. 18, 2002; U.S. Ser. No. 10/034,451, filed Dec. 28, 2001; International application no. PCT/US01/10071, filed Mar. 28, 2001; International application no. PCT/US01/46418, filed Dec. 7, 2001; and International application no. PCT/US02/16382, filed May 22, 2002, all which are incorporated by reference in their entirety. Metal nanoparticles >30 nm diameter are preferred for homogenous detection of probe-target analyte complexes on an illuminated waveguide. Metal nanoparticles >30 nm diameter are known to scatter light with high efficiency, where the scattering intensity scales with the sixth power of the radius for individual particles. Further, the surface plasmon band frequency of metal nanoparticles, which leads to the absorbance and scattering of specific wavelengths of light, is dependent on particle size, chemical composition, particle shape, and the surrounding medium, such that a decrease in interparticle distance between two or more metal nanoparticles results in changes in the surface plasmon band frequency and intensity. For example, when two metal nanoparticle particles with specific binding members bind to adjacent regions of a target analyte, a change in the surface plasmon band frequency occurs leading to a change in solution color. Metal nanoparticles in the size range of 40-80 nm diameter are most preferred since monodisperse particles (<15% CV) can be synthesized, and the changes in the color and intensity of scattered light can be monitored visually or with optical detection instrumentation on an illuminated waveguide. A variety of metal nanoparticle compositions also could be used in the reported invention including gold, silver, copper, and other metal particles well known in the art or alloy or core-shell particles. For example, a core-shell particle can be a nanoparticle having a metal or non-metal (e.g. silica or polystyrene) core coated with a shell of metal. Such core-shell particles are described, for example, in Halas et al., 1999, Applied Physics Letters 75:2197-99 and Halas et al., 2001, J of Phys Chem. B 105:2743, which is incorporated by reference herein in its entirety. In one embodiment, other types of metal nanostructures that have a surface plasmon band can be used in the methods of the invention. The most preferred particle composition is gold since it is highly stable and can be derivatized with a variety of biomolecules. The most preferred particle and size range is 40-80 nm diameter gold particles.

When using dextran sulfate to drive the formation of nanoparticle probe-target analyte complexes, the preferred detection embodiment is an illuminated waveguide, which enables the monitoring of scattered light from the complexes within the penetration depth of the evanescent field. In addition to high detection efficiency associated with monitoring nanoparticle scatter, which is well known in the art, the formation of metal nanoparticle probe-target complexes not only leads to a shift in color, but also provides a substantial increase in the intensity of light scattered when compared to an uncomplexed metal nanoparticle probe.

Unlike previously reported systems, this enables homogeneous detection of target analytes in the presence of an excess of nanoparticle probes. An example is two 50 nm gold probes bound to a DNA target, where a visually detectable color change is observed on the waveguide in the presence of up to 20 fold excess of unbound gold nanoparticle probes after the sample is dried onto the waveguide (note that the sample may not be fully dried as dextran sulfate retains some moisture under some conditions), without removing the excess unbound gold nanoparticle (i.e. homogeneous reaction). As a result, homogeneous detection of target analyte can be driven with an excess of nanoparticle probe, and in conjunction with dextran sulfate enables femtomolar concentrations of target analyte (e.g. specific genomic DNA sequences) to be detected with picomolar concentrations of 50 nm diameter gold probe.

In addition, the detectable probe/target ratio can be increased substantially by using more than two probes that bind to a target analyte. By binding four 50 nm gold probes to adjacent regions of a DNA target in the homogeneous assay, over 200 fold excess of gold nanoparticle probe can be used in the methods of the invention, and a change in colorimetric scatter is still detectable on an illuminated waveguide. By using an excess of probe to target, significantly lower concentrations of target analyte can be detected with the methods of the invention either visually or with optical detection instrumentation.

Scattered light can be detected visually or by photoelectric means. For visual detection, the observer visually determines whether or not scattering has occurred at a discrete region. For instance, scattering is observed when the discrete region appears brighter than the surrounding background or a control spot that contains uncomplexed particles located at an adjacent region. Alternatively, the observer can determine what color of light is scattered at a discrete region. For instance, a scatter color of orange at a discrete region of interest can be compared to the surrounding background or to a control spot containing uncomplexed particles that scatters no light or weak green light depending on particle size located at an adjacent region. If there are numerous discrete regions, a photoelectric detection system is preferred. Photoelectric detection systems include any system that uses an electrical signal which is modulated by the light intensity and/or frequency at the discrete region.

There are a number of avenues with different modes of illumination and imaging that are demonstrated herein for the detection of gold nanoparticle complexes on transparent substrates for the purposes of biomolecule or molecular detection. In the first method, planar illumination of a transparent substrate with white light generates an evanescent wave on the slide surface, and the light scattered from samples on the substrate is collected with a monochrome photosensor (e.g. CMOS or CCD). In the second method, planar illumination of a transparent substrate with white light generates an evanescent wave on the slide surface, and the light scattered from samples on the substrate is collected with a color photosensor (e.g. CMOS or CCD). In the third method, planar illumination of a transparent substrate with a specific wavelength of light generates an evanescent wave at the slide surface, and the light scattered from samples on the substrate is collected with a monochrome or color photosensor. An alternative method is planar illumination of a transparent substrate with white light, which generates an evanescent wave at the slide surface, and the light scattered from samples on the substrate is filtered with a specific wavelength filter and collected onto a monochrome photosensor. In addition, the light scattered from probe complexes formed in the presence of neutral or anionic polysaccharide can be monitored using non-evanescent scattering techniques. The light scattered from probe complexes also may be detected using a diode array detector.

In one embodiment, as shown in representative FIG. 1, the present invention relates to a method for detecting for the presence of one or more target analytes in a sample, each target analyte having at least two binding sites for specific binding interactions with specific binding complements, in a sample, utilizing:
a. at least one type of capture substrates, the captures substrates having bound thereto at least one first capture moiety, wherein the first capture moiety is a specific binding complement of the specific target analyte that binds to at least a first binding site of the specific target analyte;
b. at least one type of nanoparticle detection probes, the nanoparticle detection probes having bound thereto i) at least one kind of non-nucleic acid receptor, and ii) at least one kind of non-nucleic acid markers, and iii) at least one second capture moiety, wherein said second capture moiety is a specific binding complement of the specific target analyte that binds to a least a first binding site of the specific target analyte (second capture moiety);
c. contacting the capture substrates, nanoparticle detection probes and samples thought to contain the specific target analyte to form a sandwich-like complex;
d. optionally isolating and washing the capture substrate to remove unbound nanoparticle detection probes; and
e. detecting for the presence of the non-nucleic acid markers, wherein the presence of the non-nucleic acid markers is indicative of the presence of the specific target analyte in the sample.

Thus, in one embodiment of the invention, the capture substrate comprises a magnetic bead or magnetic rod or bar. This capture substrate is coated with a first capture moeity, which comprises at least one specific binding complement of the target analyte. For example, if the target analyte is a protein, the first capture moeity would be a target protein-specific antibody, as shown in FIG. 2A. If the target analyte is nucleic acid, the capture moeity may be a nucleic acid complementary to at least one portion of the target nucleic acid analyte, as shown in FIG. 2B.

In another embodiment, the invention utilizes a type of novel nanoparticle detection probes which comprises nanoparticles which optionally have bound thereto at least one kind of non-nucleic acid marker, preferably a large number of said non-nucleic acid markers, as shown in representative FIGS. 1 and 2. In another embodiment of the present invention, microparticles may be used.

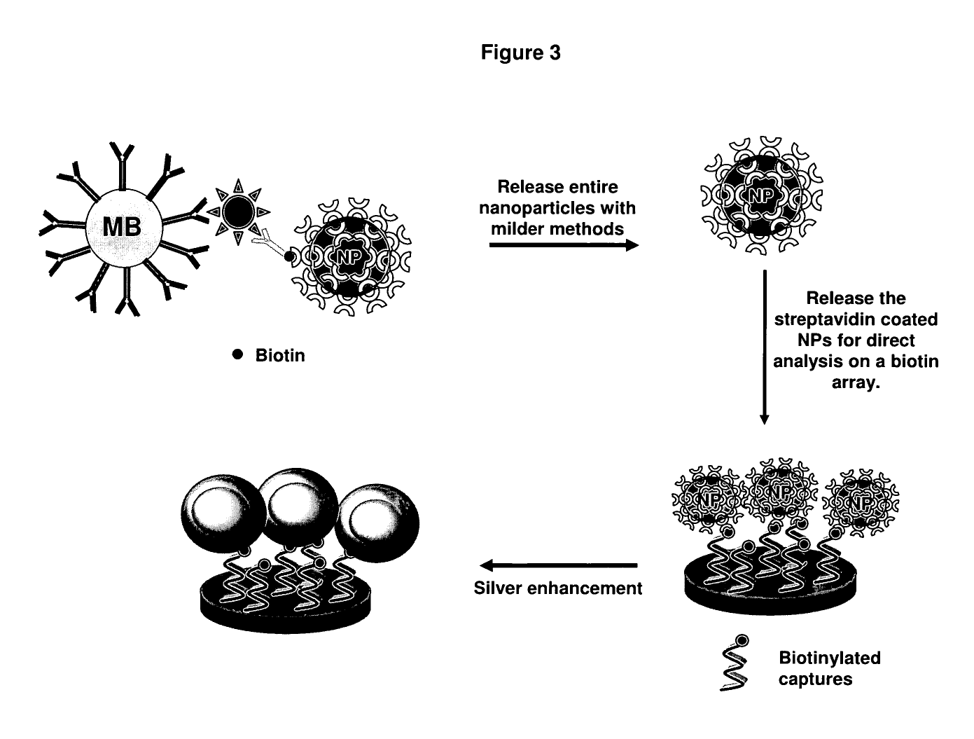

Said non-nucleic acid markers comprise two marker molecules linked by a non-nucleic acid linker, wherein each marker is at least one member of a specific binding pair, such as biotin, DIG, or DNP. Alternatively, the non-nucleic markers may comprise one member of a specific binding pair, directly bound to another member of a specific binding pair. For instance, a analyte-specific antibody may be directly bound to biotin, as shown in FIG. 3. Said non-nucleic acid markers are bound to nanoparticles either directly, or via specific binding pairs. For example, if one marker of the non-nucleic acid marker is biotin, the nanoparticle may be coated with streptavidin, as shown in FIG. 2A; if one marker molecule of the non-nucleic acid marker is DIG, the nanoparticle may be coated with anti-DIG antibody, as shown in FIG. 1B; if the marker molecule of the non-nucleic acid marker is DNP, the nanoparticle may be coated with anti-DNP antibody, as shown in FIG. 1A. The two marker molecules of said non-nucleic acid markers may be the same, or may be different. Furthermore, the linker may be any molecule that does not interfere with the binding of the non-nucleic acid markers to their corresponding receptors. For instance, the linker may be an olefin, a polymer, a cholesterol structure, a modified teflon, or carbohydrate.

Alternatively, the nanoparticle may be directly loaded with one member of a specific binding pair, for example biotin or streptavadin, as in FIGS. 2A and 2B.

The nanoparticle detection probe may further comprise a second capture moiety, as shown in FIG. 1, for example. This second capture moiety comprises at least one specific binding complement of the target analyte. The second capture moeity may be any compound capable of selectively recognizing and binding to the target analyte without interfering with the binding between the target analyte and the first capture moiety. Example of suitable selective binding compounds include, but are not limited to, antibodies, enzymes, proteins, oligonucleotides and inorganic compounds. This capture moeity may optionally be labeled with a detectable marker, such as biotin, florescence, radioactivity, etc., as in FIGS. 3, 4 and 5, for example.

The preferred detection method utilizing this amplification material is similar to that used in a sandwich immunoassay. In particular, the sample being analyzed is exposed to a capture substrate capable of selectively and specifically binding to species of interest, the capture substrate being comprised of a capture moiety immobilized on an insoluble material, such as a magnetic bead. Any unbound materials are then separated from the immobilized analyte through standard means. Immobilized analyte is then exposed to the detection probe of this invention. The detection reagent binds to the immobilized analyte through the selective binding moieties incorporated thereon. The “sandwich” complex structure thus formed (capture substrate-analyte-detection probe) therefore effectively immobilizes the detection reagent on the insoluble substrate. Unbound detection reagent can be separated from this immobilized structure through standard methods. Amplification is performed by exposing the immobilized insoluble substrate-analyte-detection reagent sandwich to some means of separating the biobarcode or non-nucleic acid marker, from sandwich complex, resulting in the release of the non-nucleic acid markers into the medium, or alternatively, the nanoparticle detection probe itself is detected.

As the ratio of the numbers of non-nucleic acid markers and non-nucleic acid marker receptors initially bound to the detection probe can be established at greater than one during preparation of the detection probe, release of the non-nucleic acid markers from a particle results in more reporter moieties entering the medium than there are target analyte molecules bound to the insoluble substrate. Detection, and optionally quantitation, of the released reporter moieties can be performed using any method that is appropriate to the chemical nature of the non-nucleic acid marker. The significant amplification of the detected signal of the non-nucleic acid marker from the detection of individual target analyte molecules results in an extremely sensitive, reliable and adaptable chemical detection assay. This ratio establishes the amplification of the signal from the detection of a target analyte molecule. For example, the release of the non-nucleic acid markers from one detection probe bearing 1000 copies of the non-nucleic acid marker that is bound to one molecule of immobilized analyte will result in 1000 molecules of non-nucleic acid marker appearing in the medium for each molecule of analyte in the original sandwich. This results in the chemical signal represented by the target analyte being amplified by a factor of 1000. This amplification can be adjusted during the synthesis of the detection probe by manipulating parameters such as the surface area of the non-nucleic acid marker and the ratio between and the packing densities of the non-nucleic acid marker receptor and non-nucleic acid marker on the surface of the detection probe. Thus, the size of the detection probe dictates the number of non-nucleic acid markers that can be released, and the ultimate amplification factor that is obtained with regard to labeled target molecules.

The non-nucleic acid marker may be attached to the surface of the detection probe by means sufficiently strong enough to prevent significant non-specific release of the non-nucleic acid marker during the steps of the detection method but simultaneously susceptible to separation and release of the non-nucleic acid marker immediately prior to the detection step. Thus, the non-nucleic acid marker may be attached to the surface of the detection probe directly through a biotin-streptavidin binding interaction that can be disrupted prior to the detection step. Alternatively, the non-nucleic acid marker may be attached to the surface of the reporter particle indirectly.

If desired, the non-nucleic acid markers may optionally include detection labels including, but are not limited to, fluorophores, chromophores, oligonucleotides with or without attached fluorophores or chromophores, proteins including enzymes and porphyrins, lipids, carbohydrates, synthetic polymers and tags such as isotopic or radioactive tags.

In the first step of the detection method of the present invention, the sample being analyzed for the presence of the target molecule is exposed to a capture substrate comprising a first capture moeity such as an antibody, oligonucleotide, lectin or similar material that is capable of selectively and specifically binding to the target specie of interest. The capture phase is immobilized on an insoluble material that is compatible with the assay chemistry and that it can readily be separated from the reaction medium. The immobilized capture phase is constructed such that it specifically binds, captures and immobilizes the analyte of interest, but preferably does not bind any other materials that may be present in the sample. Examples of the insoluble material suitable for use in the methods of the present invention include, but are not limited to, wells of a microtiter plate, a nanoparticle, fibrous or membrane filters, or other such insoluble materials. The preferred insoluble material is a magnetic particle.

The first capture moiety is preferably selected such that it binds to a different determinant on the analyte than does the second capture moeity component of the detection probe. Any unbound materials are then separated from the immobilized target analyte by any suitable means including, for example, decantation, sedimentation, washing, centrifuging or combinations of these processes. The net result of this process is that the analyte of interest is present in a purified and concentrated state on the surface of the insoluble material.

In a subsequent step of the method of the present invention, the immobilized target analyte is exposed to the detection probe of this invention such as the streptavidin-biotin complex or nanoparticle coated with non-nucleic acid marker receptors and non-nucleic acid markers and at least a second capture moeity which selectively binds the target analyte. The second capture moeity specifically binds to the target analyte forming a “sandwich” structure including the insoluble capture substrate bound to the target analyte which is, in turn, bound to the detection probe. This sandwich structure effectively immobilizes the detection probe on the insoluble substrate, and any unbound detection probe can be separated from this immobilized structure by any suitable methods such as decantation, sedimentation, washing, centrifuging or combinations of these processes as noted above.

In another step of the present method the signal from the binding and detection of the target analyte is amplified by exposing the immobilized insoluble capture substrate-target analyte-detection probe sandwich to conditions that can liberate the non-nucleic acid marker from the detection probe. The liberated non-nucleic acid marker then enters the media surrounding the detection probe bound to the target analyte as described in detail above.

The media containing the released reporter moiety may be analyzed for the presence of the released non-nucleic acid markers using any method that is appropriate to the chemical nature of the non-nucleic acid marker. For example, a fluorescently-labeled non-nucleic acid marker may be detected and even quantitated by measurement of the fluorescence intensity or fluorescence depolarization of the medium while the presence of a chemiluminescent-labeled reporter can be determined by measuring the luminescence that occurs upon addition of an appropriate trigger reagent. Numerous other options including electrochemical, impedance, enzymatic and radioactivity detection are also available.

In another embodiment of the present invention, the capture substrates, target analyte, and nanoparticle detection probes are added consecutively. In another embodiment of the present invention, the capture substrates, target analytes, and nanoparticle detection probes are added simultaneously. In a further embodiment, target analytes are added to capture substrates, and nanoparticle detection probes are added subsequently. In a further embodiment, target analytes are added to nanoparticle detection probes, and capture substrates are added subsequently.

In yet another embodiment, the first capture moiety is a target analyte-specific antibody. Alternatively the first capture moiety is a nucleic acid, the sequence of which is complementary to at least one portion of the sequence of the target analyte nucleic acid, as shown in FIG. 2B. In another embodiment, the second capture moiety is a specific binding complement of the specific target analyte, that binds to at least a second binding site of the specific target analyte, as in FIGS. 1, 2 and 3, for example.

In a further embodiment, the second capture moiety comprises a label, wherein said label is biotin, DIG, DNP or streptavidin.

In yet another embodiment, the non-nucleic acid receptors are anti-DIG antibodies, anti-DNP antibodies, biotinylated target-specific antibodies, or streptavidin. The non-nucleic acid markers may be comprised of biotin, DNP or DIG. In alternate embodiments, the component markers of non-nucleic acid markers may be different, as in FIGS. 1A and 1B, or may be the same marker, as in FIG. 2B.

In yet a further embodiment, microparticle detection probes may be used, rather than nanoparticle detection probes, where the microparticles may be between 1 and 5 micrometers in size. In a preferred embodiment, the nanoparticles are between 5 and 200 nm in size.

After binding of the non-nucleic acid marker-bound nanoparticle to the analyte and capture substrate, the non-nucleic acid markers may be released using appropriate denaturing or release methods.

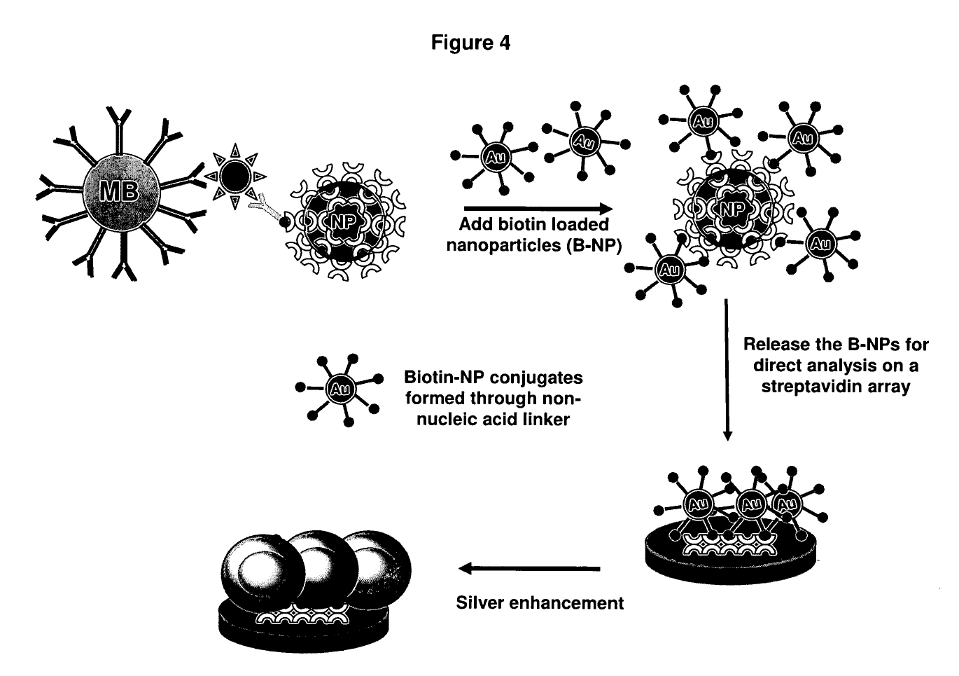

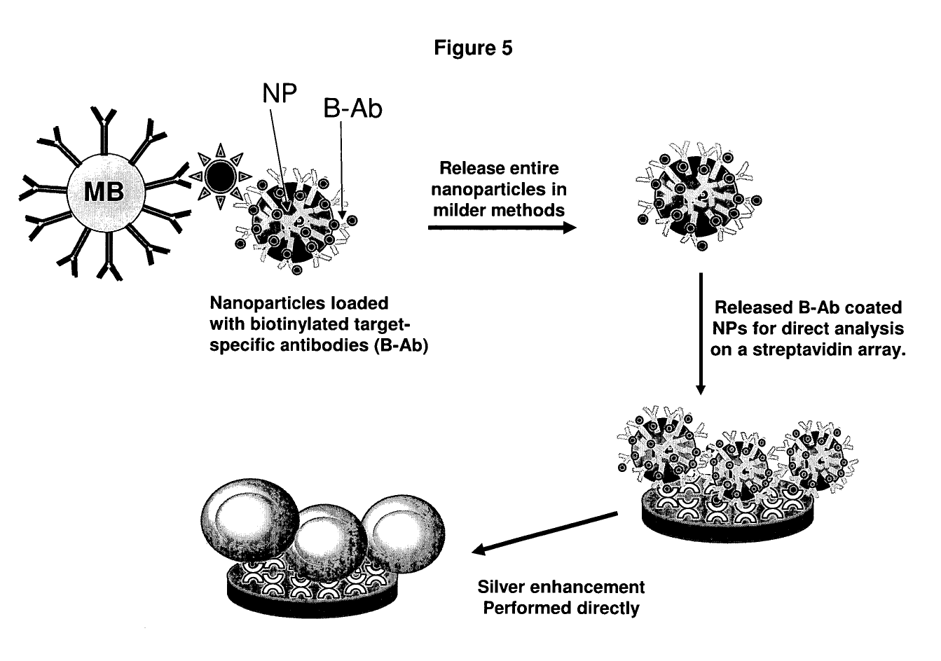

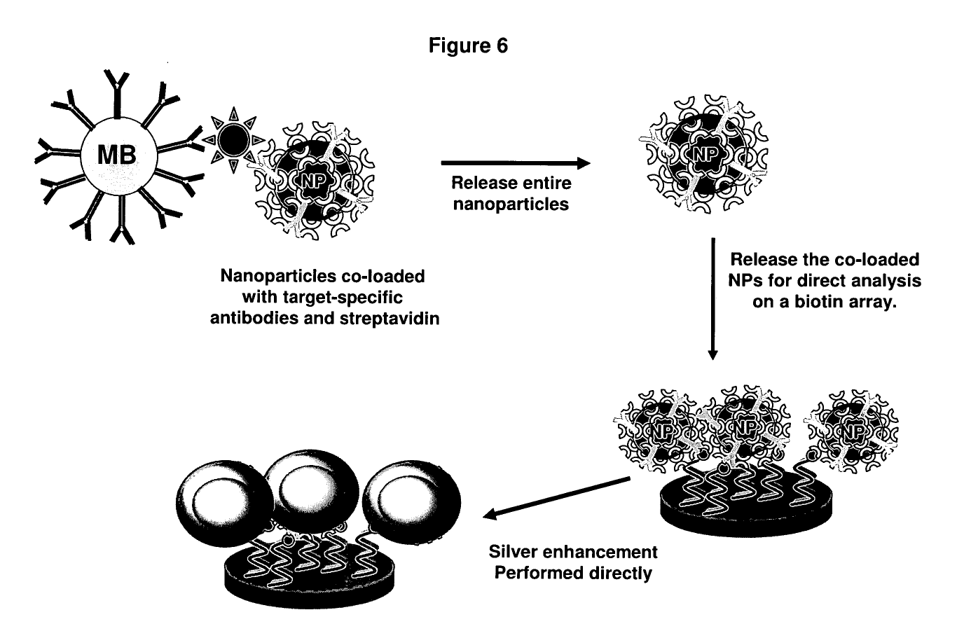

In the detection step, the non-nucleic acid markers are captured on a solid substrate, and detected with gold nanoparticles after silver enhancement. Alternatively, the nanoparticle itself, if coated with streptavidin, could be detected by biotinylated captures attached to a detection substrate, as shown in FIG. 3. In a further embodiment, a streptavidin-coated nanoparticle will be released from the non-nucleic acid marker-analyte-capture substrate complex; then, biotin-loaded nanoparticles could be added. After separating the nanoparticles from the rest of the solution, the biotin-labeled nanoparticles may be detected on a streptavidin array, as shown in FIG. 4. Alternatively, the nanoparticle detection probe itself may be directly labeled with biotinylated antibodies, or another non-nucleic acid receptor; after washing, the entire nanoparticle can be detected on a streptavidin array, as shown in FIG. 5. In a further embodiment, the nanoparticle may be coated with anti-analyte antibodies and streptavidin. Once released, the nanoparticles may be detected on a biotinylated array, as shown in FIG. 6.

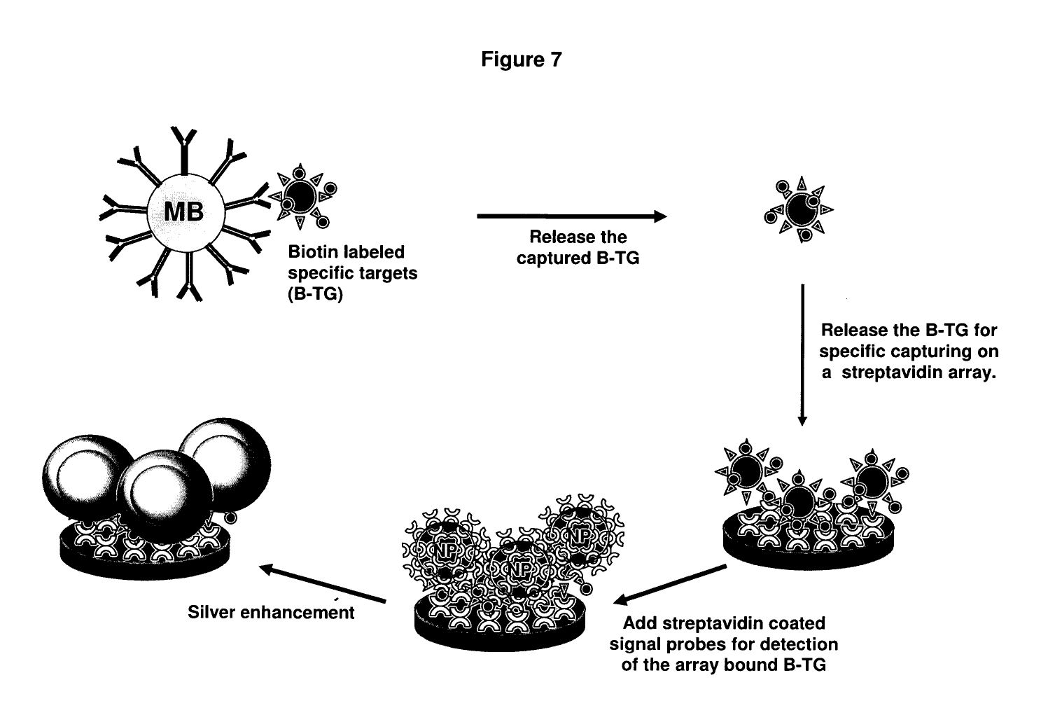

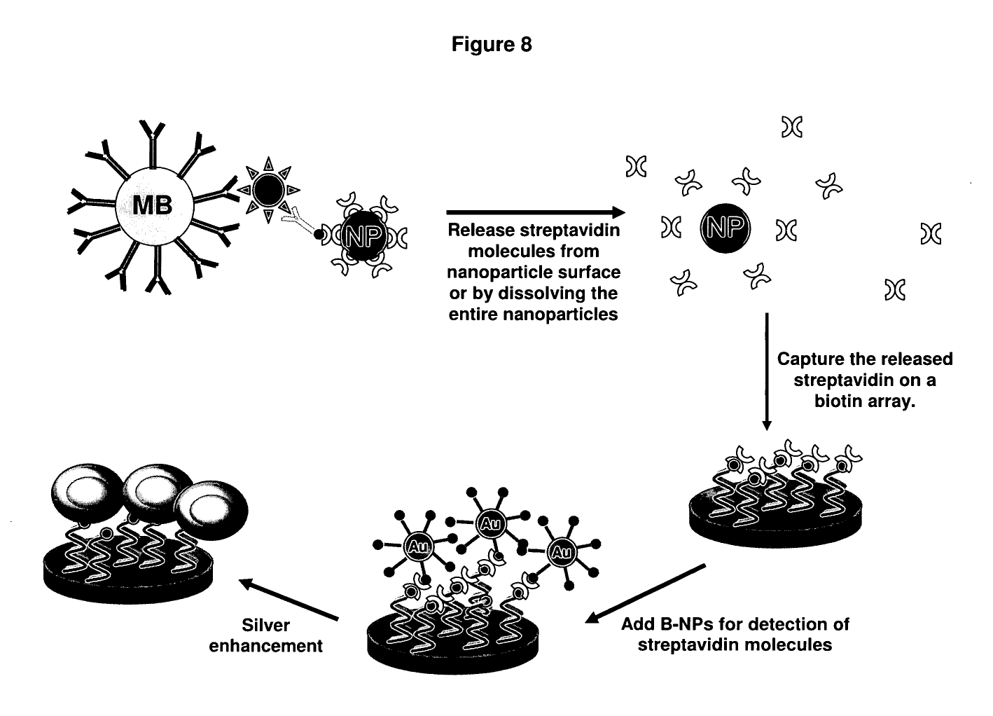

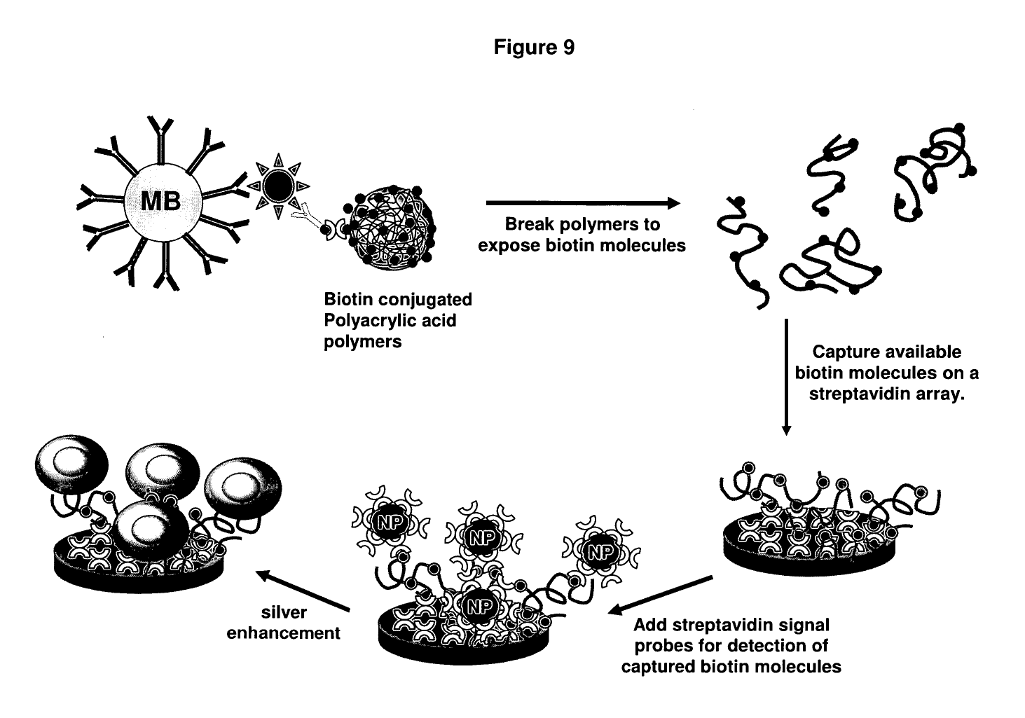

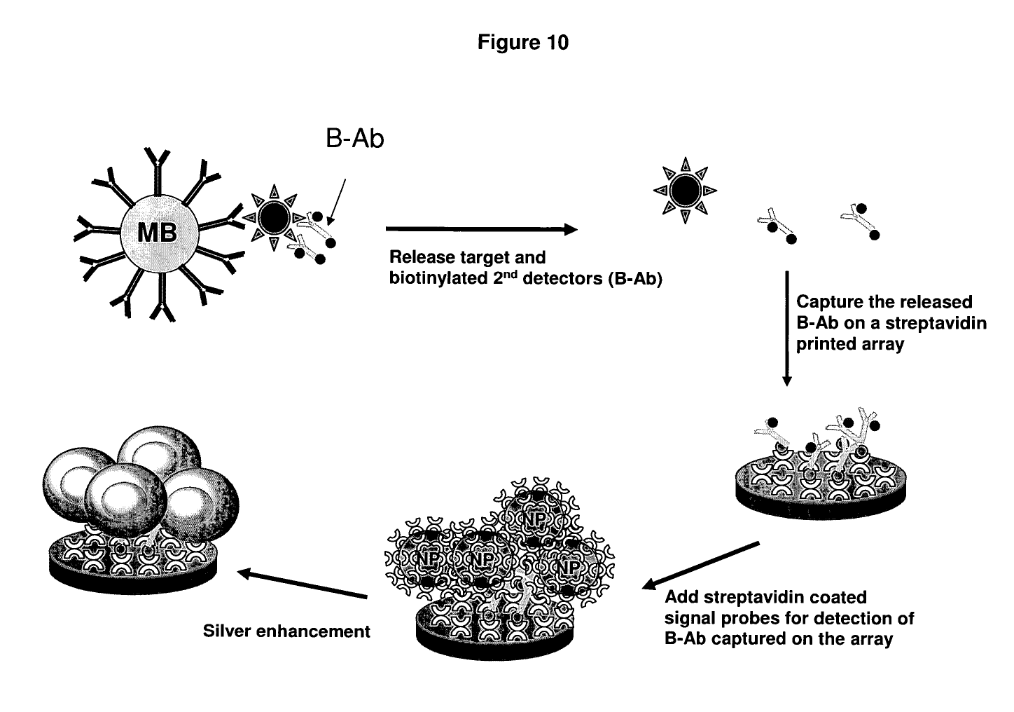

In a further embodiment, the biotin-labeled target may be directly detected on a streptavidin array, after washing, as shown in FIG. 7. Alternatively, a streptavidin-coated nanoparticle can serve as the nanoparticle detection probe; in this case, after washing, the nanoparticle is released, then dissolved, releasing streptavidin, which may then be captured on a biotinylated array. Biotin-loaded gold nanoparticles may then be added to detect the presence of streptavidin, as shown in FIG. 8. In another embodiment, the detection probe is biotin-conjugated polyacrylic acid polymers, which, after washing, are dissociated to expose the biotin, which is then captured on a streptavidin array. Streptavidin-coated nanoparticles are added to detect the presence of the biotin-conjugated polymers, as shown in FIG. 9. In one further embodiment, the non-nucleic acid markers themselves serve as a detection probe; biotinylated antibodies can be captured on a streptavidin array, and streptavidin-coated nanoparticles can be added to detect the presence of biotinylated antibodies, as shown in FIG. 10.

It is to be noted that the term “a” or “an” entity refers to one or more of that entity. For example, “a target analyte” refers to one or more target analyte or at least one target analyte. As such, the terms “a” (or “an”), “one or more” and “at least one” are used interchangeably herein. It is also to be noted that the terms “comprising”, “including”, and “having” have been used interchangeably.

### EXAMPLES

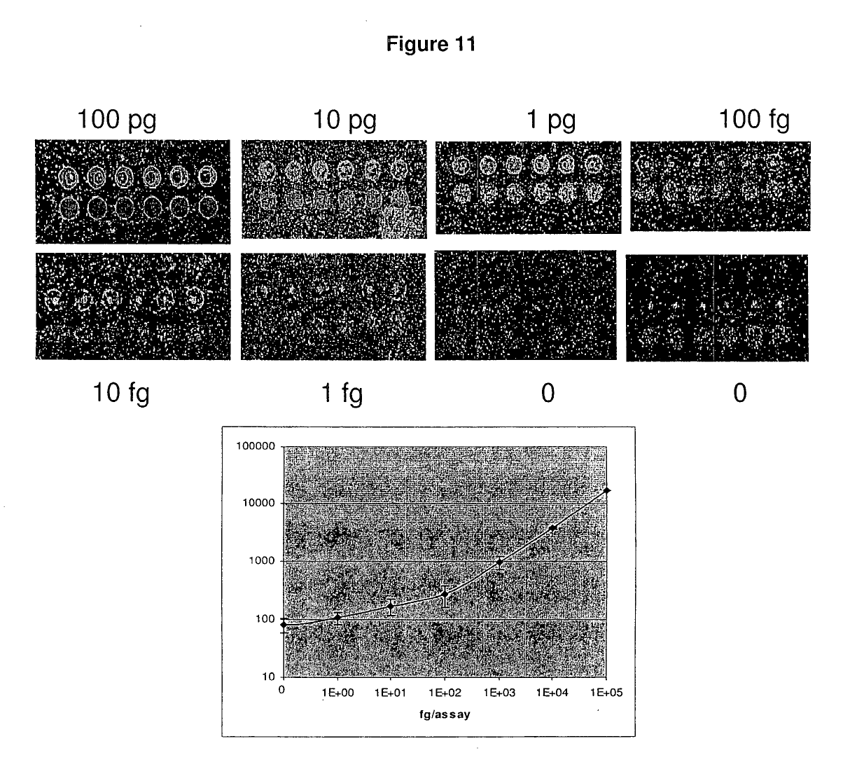

The following examples are offered to illustrate, but not to limit, the invention. FIGS. 3 and 11 illustrate the examples below.

### Example 1

FIG. 3 shows a schematic of the example. Nanoparticle detection probe is coated with streptavidin and has biotin-labeled target-specific second capture moieties, in this case, antibodies, bound thereto. The capture substrate shown in FIG. 3 is a magnetic bead coated with a first target-specific capture moiety, in this case, antibodies. After removing the complexes from the sample solution, and washing unbound nanoparticle detection probes, the nanoparticle detection probes are released from the complex. The released nanoparticle detection probes are then bound to a substrate coated with biotinylated captures, and detected with nanoparticle probes after silver amplification. The presence of the nanoparticle detection probe indicates the presence of the target analyte in the sample.

The assay conditions were typically performed in 1×PBS buffer, pH 7.5, 0.1% BSA, and 0.025% Tween 20. Each binding step in the sandwich (complex) formation was carried out at 25° C. on a shaker (˜1200 rpm) for efficient mixing. The duration for the target binding to the first capture moiety was 30-60 min, for the second capture moiety binding to bound targets was 30 min, and for streptavidin coated nanoparticle detection probe binding was 30 min.

### Example 2

Prostate Specific Antigen (PSA) target detection is used as an example of this invention. PSA target was tested from 100 pg, 10 pg, 1 pg, 100 fg, 10 fg, 1 fg to 0 fg per assay. Different amounts of target was first captured using 2 μg of magnetic beads (MB) [Dynabeads® Myone™ Tosylactivated, coated with PSA antibody [Biodesign, MAb, α-PSA free form, Cat#M86806M, Lot #21k31504, clone #8A6] in 200 uL of Barcode Buffer (1×PBS [Gibco, Cat #70013-032, Lot#1148371] 0.5% BSA [R&D System, Cat# Dy995, part#841380, Lot#225340], 0.05% Tween 20 [SigmaUltra, P-7949, Lot#81K0293]) at 25° C. with shaking at 200 rpm for 90 minutes. To form a specific sandwich, 100 ng of the biotinylated anti-human Kallikrein 3 polyclonal goat IgG [anti-PSA-biotin AB, R&D System cat#BAF1344, Lot#IR013071] is added as a secondary antibody and incubated for an additional one hour at 25° C. with shaking at 1200 rpm. After two times washing with Barcode Buffer, 1 μL of the streptavidin coated nanoparticles. The bound streptavidin coated nanoparticles (a component of the specific complex) are released and applied to a biotin printed microarray. Array binding reaction was performed in 50 μL buffer (1×PBS, 0.025% Tween 20, 0.05% BSA) incubated at 25° C. with shaking at 1200 rpm for 1 hour. After washing with 0.5N NaNO₃ four times, array was developed with silver and signals measured with light scattering. The scanned image and data analysis were shown in FIG. 11. The assay conditions were the same as in Example 1.

It should be understood that the foregoing disclosure emphasizes certain specific embodiments of the invention and that all modifications or alternatives equivalent thereto are within the spirit and scope of the invention as set forth in the appended claims.

### REFERENCES

(1) A. Pandey and M. Mann, Nature 2000, 405, 837-846.
(2) S. Fields and O. K. Song, Nature 1989, 340, 245-246.
(3) M. Ijksma, B. Kamp, J. C. Hoogvliet, W. P. van Bennekom, Anal. Chem. 2001, 73, 901-907.
(4) R. F. Service, Science, 2000, 287, 2136-2138.
(5) H. Zole, Monoclonal Antibodies, Springer-Verlag, New York, 2000, p. 1-5.
(6) J. E. Butler, J. Immunoassay, 2000, 21(2 & 3), 165-209.
(7) P. Herbrink, A. Noorduyn, W. C. Van Dijk, Tech. Diagn. Pathol. 1991, 2, 1-19.
(8) C. A. Mirkin, R. L. Letsinger, R. C. Mucic, J. J. Storhoff, Nature 1996, 382, 607-609
(9) J. J. Storhoff, C. A. Mirkin, Chem. Rev. 1999, 99, 1849-1862.
(10) S.-J. Park, A. A. Lazarides, C. A. Mirkin, P. W. Brazis, C. R. Kannewurf, R. L. Letsinger, Angew. Chem. Int. Ed. 2000, 39, 3845-3848.
(11) T. A. Taton, C. A. Mirkin, and R. L. Letsinger, Science 2000, 289, 1757-1760.
(12) S.-J. Park, A. A. Lazarides, C. A. Mirkin, and R. L. Letsinger, Angew. Chem. Int. Ed. 2001, 40, 2909-2912.
(13) Z. Eshhar, M. Ofarim, and T. Waks, J. Immunol. 1980, 124, 775-780.
(14) M. Wilcheck and E. A. Bayer, Immunol. Today 1984, 5, 39-43
(15) N. Winssinger, J. L. Harris, B. J. Backes, P. G. Schultz, Agnew. Chem. Int. Ed. 2001, 40, 3152-3155
(16) G. MacBeath, A. N. Koehler, S. L. Schreiber, J. Am. Chem. Soc. 1999, 121, 7967-7968.
(17) P. J. Hergenrother, K. M. Depew, S. L. Schreiber, J. Am. Chem. Soc. 2000, 122, 7849-7850.
(18) J. J. Storhoff, R. Elghanian, R. C. Mucic, C. A. Mirkin, R. L. Letsinger, J. Am. Chem. Soc. 1998, 120, 1959-1964.
(19) R. C. Mucic, J. J. Storhoff, C. A. Mirkin, R. L. Letsinger, J. Am. Chem. Soc. 1998, 120, 12674-12675.
(20) L. M. Demers, C. A. Mirkin, R. C. Mucic, R. A. Reynolds III, R. L. Letsinger, R. Elghanian, G. Viswanadham, Anal. Chem. 2000, 72, 5535-5541.
(21) T. Brown, D. J. S. Brown, in Oligonucleotides and Analogues (Ed.: F. Eckstein), Oxford University Press, New York, 1991.
(22) L. A. Chrisey, G. U. Lee, and C. E. O'Ferral, Nucl. Acids. Res. 1996, 24, 3031-3039.
(23) Nicewamer-Pena, S. R. Freeman, R. G.; Reiss, B. D.; He, L.; Pena, D. J.; Walton, I. D.; Cromer, R.; Keating, C. D.; Natan M. J. Science 2001, 294, 137.
(24) Ferguson, J. A.; Steemers, F. J.; Walt, D. R. Anal. Chem. 2000, 72, 5618.
(25) Han, M.; Gao, X.; Nie, S, Nature biotech. 2001, 19, 631.
(26) R. K. Saiki et al., Science 1985, 230, 1350.
(27) R. K. Saiki et al., Science 1988, 239, 487.
(28) R. A. Gibbs, Curr. Opin. Biotechnol. 1991, 2, 69.
(29) S. Bortolin, T. K. Christopoulos, M. Verhaegen, Anal. Chem. 1996, 68, 834.
(30) B. Deiman, P. van Aarle, P. Sillekens, Mol. Biotechnol. 2002, 20, 163.
(31) S. E. Stiriba, H. Frey, R. Haag, Angew. Chem. Int. Ed. 2002, 41, 1329.
(32) S. A. Bustin, Journal of Molecular Endocrinology 2002, 29, 23.
(33) G. MacBeath, S. L. Schreiber, Science 2000, 289, 1760.
(34) H. Zhu et al., Science 2001, 293, 2101.
(35) B. B. Haab, M. J. Dunham, P. O. Brown, Genome Biol. 2001, 2(2): RESEARCH 0004.1.
(36) J.-M. Nam, S.-J. Park, C. A. Mirkin, J. Am. Chem. Soc. 2002, 124, 3820.
(37) T. Sano, C. L. Smith, C. R. Cantor, Science 1992, 258, 120.
(38) V. Ruzicka, W. Marz, A. Russ, W. Gross, Science 1993, 260, 698.
(39) A. McKie, D. Samuel, B. Cohen, N. A. Saunders, J. Immunol. Methods 2002, 270, 135.
(40) H. Zhou, R. J. Fisher, T. S. Papas, Nucl. Acids Res. 1993, 21, 6038.
(41) E. R. Hendrickson, T. M. Hatfield-Truby, R. D. Joerger, W. R. Majarian, R. C. Ebersole, Nucl. Acids Res. 1995, 23, 522.
(42) C. M. Niemeyer et al., Nucl. Acids Res. 1999, 27, 4553.
(43) B. Schweitzer et al., Proc. Natl. Acad. Sci. U.S.A. 2000, 97, 10113.
(44) C. M. Neimeyer, R. Wacker, M. Adler, Angew. Chem. Int. Ed. 2002, 40, 3169.
(45) C. M. Niemeyer, Trends Biotechnol. 2002, 20, 395.
(46) H. Yu, E. P. Diamandis, A. F. Prestigiacomo, T. A. Stamey, Clin. Chem. 1995, 41, 430.
(47) W. J. Catalona, et al., J. Am. Med. Assoc. 1995, 274, 1214.
(48) J. L. Stanford, et al., Prostate Cancer Trends 1973-1995, SEER Program, National Cancer Institute. NIH Pub. No. 99-4543. Bethesda, Md., 1999.
(49) J. Moul, J. Urol. 2000, 163, 1632.
(50) L. A. Chrisey, G. U. Lee, C. E. Oferral, Nucl. Acids Res. 1996, 24, 3031.
(51) Y. C. Cao, R. Jin, C. A. Mirkin, Science 2002, 297, 1536-1540.
(52) R. K. Saiki et al., Science 230, 1350 (1985).
(53) S. A. Bustin, Journal of Molecular Endocrinology 29, 23 (2002).
(54) M. U. Kopp, A. J. de Mello, A. Manz, Science 280, 1046 (1998).
(55) T. H. Rider et al., Science 301, 213 (2003).
(56) Y. W. Tang, G. W. Procop, D. H. Pershing. Clin. Chem. 43, 2021 (1997).
(57) G. M. Makrigiorgos, S. Chakrabarti, Y. Zhang, M. Kaur, B. D. Price, Nat. Biotechnol. 20, 936 (2002).
(58) W. P. Halford, Nat. Biotechnol. 17, 835 (1999).
(59) R. Sutthent et al., J. Clin. Microbiol. 41, 1016 (2003).
(60) B. Schweitzer, S. Kingsmore, Curr. Opin. Biotechnol. 12, 21 (2001).
(61) S. R. Nicewamer-Pena et al., Science 294, 137 (2001).
(62) M. Han, X. Gao, S, Nie, Nat. Biotechnol. 19, 631 (2001).
(63) X. Zhao, R. Tapec-Dytioco, W. Tan, J. Am. Chem. Soc. 125, 11474 (2003).
(64) L. A. Lortie et al., J. Clin. Microbiol. 29, 2250 (1991).
(65) L. R. Zeph, X. Y. Lin, G. Stotzky, Curr. Microbiol. 22. 79 (1991).
(66) C. J. Yu et al., J. Am. Chem. Soc., 123, 11155 (2001).
(67) T. A. Taton, C. A. Mirkin, R. L. Letsinger, Science 289, 1757 (2000).
(68) S.-J. Park, T. A. Taton, C. A. Mirkin, Science 295, 1503 (2002).
(69) Y. C. Cao, R. Jin, C. A. Mirkin, Science 297, 1536 (2002).
(70) A. Saghatelian, K. M. Guckian, D. A. Thayer, M. R. Ghadiri, J. Am. Chem. Soc. 125, 344 (2003).
(71) J.-M. Nam, C. S. Thaxton, C. A. Mirkin, Science 301, 1884.
(72) J.-M. Nam, S.-J. Park, C. A. Mirkin, J. Am. Chem. Soc. 124, 3820 (2002).
(73) L. M. Demers et al., Anal. Chem. 72, 5535 (2000).

What is claimed is:

1. A nanoparticle probe for detecting for the presence of a target analyte in a sample, wherein the target analyte has at least two binding sites, the probe comprising a nanoparticle having bound thereto:
(i) a first member of a first specific binding pair;
(ii) a capture probe comprising a specific binding complement of the target analyte labeled with a second member of the first specific binding pair; and
(iii) a reporter comprising a non-nucleic acid linker having two ends, a second member of a first specific binding pair bound to the first end of the linker and a first member of a second specific binding pair bound to the second end of the linker, wherein the reporter and capture probe are bound to the first member of the first specific binding pair.

2. The probe of claim 1, wherein the first specific binding pair comprises DNP/anti-DNP antibody or DIG/anti-DIG antibody.

3. The probe of claim 1, wherein the non-nucleic acid linker comprises a polymer, 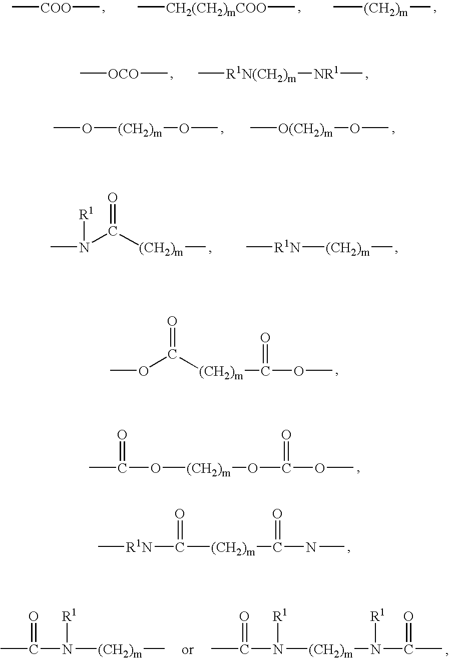
wherein:
R¹ has the formula X (CH₂)ₘ;
X is —CH₃, —CHCH₃, —COOH, —CO₂(CH₂)ₘCH₃, —OH, —CH₂OH, ethylene glycol, hexa(ethylene glycol), —O(CH₂)ₘCH₃, —NH₂, —NH(CH₂)ₘNH₂, halogen, glucose, maltose, fullerene C60, or a cyclic olefin; and
m is 0-30.

4. The probe of claim 1, wherein the second specific binding pair is biotin/streptavidin or biotin/avidin.

5. The probe of claim 1, wherein the nanoparticles are metal nanoparticles or semiconductor nanoparticles.

6. The probe of claim 5, wherein the nanoparticles are gold nanoparticles.

7. The probe of claim 1, wherein the first and second specific binding pairs are independently an antibody and an antigen, a receptor and a ligand, an enzyme and a substrate, a drug and a target molecule, or two strands of at least partially complementary oligonucleotides.

8. The probe of claim 1, wherein the target has more than two binding sites.

9. The probe of claim 1, wherein at least two types of probes are provided, the first type of probe having a specific binding complement to a first binding site on the target analyte and the second type of probe having a specific binding complement to a second binding site on the target analyte.

10. The probe of claim 8, wherein a plurality of types of probes are provided, each type of probe having a specific binding complement to different binding sites on the target analyte.

11. The probe of claim 1, wherein the specific binding complement and the target analyte are members of a specific binding pair.

12. The probe of claim 11, wherein members of the specific binding pair comprise nucleic acid, oligonucleotide, peptide nucleic acid, polypeptide, antibody, antigen, carbohydrate, protein, peptide, amino acid, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, or metabolites of or antibodies to any of the above substances.

13. The probe of claim 12 wherein nucleic acid and oligonucleotide comprise genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, or natural and synthetic nucleic acids.

14. The probe according to claim 1, wherein the target analyte is a nucleic acid and the specific binding complement is an oligonucleotide.

15. The probe according to claim 1, wherein the target analyte is a protein or hapten and the specific binding complement is an antibody comprising a monoclonal or polyclonal antibody.

16. The probe according to claim 1, wherein the target analyte is a sequence from a genomic DNA sample and the specific binding complements are oligonucleotides, the oligonucleotides having a sequence that is complementary to at least a portion of the genomic sequence.

17. The probe of claim 16, wherein the genomic DNA is eukaryotic, bacterial, fungal or viral DNA.

18. The probe according to claim 1, wherein the specific binding complement and the target analyte are members of an antibody-ligand pair.

19. The probe according to claim 1, wherein in addition to its first binding site, the target analyte has been modified to include a second binding site.

20. A method for detecting for the presence or absence of a target analyte in a sample, wherein the target analyte has at least two binding sites, the method comprising:
(a) providing a first substrate and a nanoparticle probe comprising a nanoparticle having bound thereto: (i) a first member of a first specific binding pair;
(ii) a capture probe comprising a specific binding complement of the target analyte labeled with a second member of the first specific binding pair; and (iii) a reporter comprising a non-nucleic acid linker having two ends, a second member of a first specific binding pair bound to the first end of the linker and a first member of a second specific binding pair bound to the second end of the linker, wherein the reporter and capture probe are bound to the first member of the first specific binding pair;
(b) immobilizing the target analyte onto the first substrate;
(c) contacting the immobilized target analyte with the nanoparticle probe under conditions effective to allow for binding between the target analyte and the nanoparticle probe to form a complex on the substrate;
(d) washing the substrate to remove unbound nanoparticle probes; and
(e) detecting for the presence or absence of the reporter, wherein the presence or absence of the reporter is indicative of the presence or absence of the target analyte in the sample.

21. The method of claim 20, wherein subsequent to step (d) and prior to step (e), further comprising step (d1) subjecting the complex to conditions effective to release the reporter.

22. The method of claim 20, wherein prior to step (e), further comprising steps (d2) capturing the reporter onto a second substrate; (d2) contacting the immobilized reporter with a second nanoparticle probe, the second nanoparticle probe having a specific binding complement to the reporter, under conditions effective to allow binding between the reporter and the second nanoparticle probe to form a complex on the second substrate; and (d3) washing the second substrate to remove any unbound second nanoparticle probe.

23. The method of claim 22, wherein step (e) detecting comprises contacting the washed second substrate with a stain.

24. The method of claim 20, wherein the nanoparticles are metal nanoparticles or semiconductor nanoparticles.

25. The method of claim 23, wherein the second nanoparticle probe is a gold nanoparticle probe.

26. The method of claim 22, wherein the second substrate is a wave guide and step (e) comprises illuminating the substrate subsequent to step (d3) and observing for any changes in the intensity of light scattered.

27. The method of claim 20, wherein the specific binding pair is an antibody and an antigen, a receptor and a ligand, an enzyme and a substrate, a drug and a target molecule, or two strands of at least partially complementary oligonucleotides.

28. The method of claim 20, wherein the target has more than two binding sites.

29. The method of claim 20, wherein at least two types of probes are provided, the first type of probe having a specific binding complement to a first binding site on the target analyte and the second type of probe having a specific binding complement to a second binding site on the target analyte.

30. The method of claim 28, wherein a plurality of types of probes are provided, each type of probe having a specific binding complement to different binding sites on the target analyte.

31. The method of claim 20, wherein the specific binding complement and the target analyte are members of a specific binding pair.

32. The method of claim 20, wherein members of a specific binding pair comprise nucleic acid, oligonucleotide, peptide nucleic acid, polypeptide, antibody, antigen, carbohydrate, protein, peptide, amino acid, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, or metabolites of or antibodies to any of the above substances.

33. The method of claim 32 wherein nucleic acid and oligonucleotide comprise genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, or natural and synthetic nucleic acids.

34. The method according to claim 20, wherein the target analyte is a nucleic acid and the specific binding complement is an oligonucleotide.

35. The method according to claim 20, wherein the target analyte is a protein or hapten and the specific binding complement is an antibody comprising a monoclonal or polyclonal antibody.

36. The method according to claim 20, wherein the target analyte is a sequence from a genomic DNA sample and the specific binding complements are oligonucleotides, the oligonucleotides having a sequence that is complementary to at least a portion of the genomic sequence.

37. The method of claim 20, wherein the genomic DNA is eukaryotic, bacterial, fungal or viral DNA.

38. The method according to claim 20, wherein the specific binding complement and the target analyte are members of an antibody-ligand pair.

39. The method according to claim 20, wherein in addition to its first binding site, the target analyte has been modified to include a second binding site.

40. A nanoparticle probe for detecting for the presence of a target analyte, wherein the target analyte is a first member of a first specific binding pair and wherein the target analyte has at least two binding sites, the probe comprising a nanoparticle having bound thereto:
(i) a first member of a second specific binding pair;
(ii) a capture probe comprising a second member of the first specific binding pair labeled with a second member of the second specific binding pair;
(iii) a reporter comprising a non-nucleic acid linker having two ends, a second member of a second specific binding pair bound to the first and second ends of the linker, wherein the reporter and capture probe are bound to the first member of the second specific binding pair.

41. The probe of claim 40, wherein the second specific binding pair is biotin/streptavidin or biotin/avidin.

42. The probe of claim 41, wherein the second member of the second specific binding pair is biotin.

43. A method for detecting for the presence or absence of a target analyte in a sample, wherein the target analyte has at least two binding sites, the method comprising:
(a) providing a first substrate and a nanoparticle probe comprising a nanoparticle having bound thereto: (i) a first member of a second specific binding pair; (ii) a capture probe comprising a second member of the first specific binding pair labeled with a second member of the second specific binding pair; (iii) a reporter comprising a non-nucleic acid linker having two ends, a second member of a second specific binding pair bound to the first and second ends of the linker, wherein the reporter and capture probe are bound to the first member of the second specific binding pair;
(b) immobilizing the target analyte onto the first substrate;
(c) contacting the immobilized target analyte with the probe under conditions effective to allow for binding interactions between the target analyte and the nanoparticle probe to form a complex on the substrate in the presence of the target analyte;
(d) washing the substrate to remove unbound nanoparticle probes; and
(e) detecting for the presence or absence of the reporter, wherein the presence or absence of the reporter is indicative of the presence or absence of the target analyte in the sample.

44. The method of claim 43, wherein subsequent to step (d) and prior to step (e), further comprising step (d1) subjecting the complex to conditions effective to release the reporter.

45. The method of claim 43, wherein prior to step (e), further comprising steps (d2) capturing the reporter onto a second substrate; (d3) contacting the immobilized marker with a second nanoparticle probe, the second nanoparticle probe having a specific binding complement to the reporter, under conditions effective to allow binding between the reporter and the second nanoparticle probe to form a complex on the second substrate; and (d4) washing the second substrate to remove any unbound second nanoparticle probe.

46. The method of claim 45, wherein step (e) detecting comprises contacting the washed second substrate with a stain.

47. The method of claim 46, wherein the second nanoparticle probe is a gold nanoparticle probe.

48. The method of claim 45, wherein the second substrate is a waveguide and step (e) comprises illuminating the substrate subsequent to step (d4) and observing for any changes in the intensity of light scattered.

49. A method for detecting for the presence or absence of a target analyte in a sample, wherein the target analyte has at least two binding sites, the method comprising:
(a) providing a first substrate;
(b) providing a first nanoparticle probe comprising a nanoparticle having (i) a first member of a first specific binding pair bound thereto and (ii) a releasable specific binding complement to the target analyte, the specific binding complement labeled with a second member of the first specific binding pair;
(c) immobilizing the target analyte onto the first substrate;
(d) contacting the immobilized target analyte with the nanoparticle probe under conditions effective to allow for binding interactions between the target analyte and the first nanoparticle probe to form a complex on the substrate in the presence of the target analyte;
(e) washing the substrate to remove unbound first nanoparticle probes;
(f) releasing the specific binding complement from the first nanoparticle probe to form a second nanoparticle probe having the first member of the first specific binding pair; and
(g) detecting for the presence or absence of the second nanoparticle probe, wherein the presence or absence of the second nanoparticle probe is indicative of the presence or absence of the target analyte in the sample.

50. The method of claim 49, wherein subsequent to step (f) and prior to step (g), further comprising steps (f1) capturing the second nanoparticle probe onto a second substrate having a second member of the first specific binding pair under conditions effective to allow binding interactions between the second nanoparticle probe and the second member of the first specific binding pair to form a complex on the second substrate in the presence of the second nanoparticle probe; and (f2) washing the second substrate to remove any unbound second nanoparticle probe.

51. The method of claim 50, wherein step (g) detecting comprises contacting the washed second substrate with a stain.

52. The method of claim 51, wherein the second nanoparticle probe is a gold nanoparticle probe.

53. The method of claim 50, wherein the second substrate is a waveguide and step (g) comprises illuminating the substrate subsequent to step (f2) and observing for any changes in the intensity of light scattered.

54. A nanoparticle probe comprising:
(i) a first nanoparticle having a first member of a non-nucleic acid specific binding pair bound thereto; and
(ii) a second nanoparticle having a non-nucleic acid linker bound thereto, the linker having a first end and a second end, wherein a the first end of the linker is bound to the second nanoparticle and the second end of the linker is bound to a second member of the specific binding pair, wherein the first and second nanoparticles are bound to each other by specific binding pair interactions.

55. The probe of claim 54, wherein the specific binding pair comprises is biotin/streptavidin or biotin/avidin.

56. The probe of claim 55, wherein the second member of the specific binding pair is biotin.

57. A method for detecting for the presence or absence of a target analyte in a sample, wherein the target analyte has at least two binding sites, the method comprising:
(a) providing a first substrate;
(b) providing a first nanoparticle probe comprising a nanoparticle having (i) a first member of a first specific binding pair bound thereto and (ii) a releasable specific binding complement to the target analyte, the specific binding complement labeled with a second member of the first specific binding pair;
(c) immobilizing the target analyte onto the first substrate;
(d) contacting the immobilized target analyte with the nanoparticle probe under conditions effective to allow for binding between the target analyte and the first nanoparticle probe to form a complex on the substrate in the presence of the target analyte;
(e) washing the first substrate to remove unbound first nanoparticle probes;
(f) releasing the specific binding complement from the first nanoparticle probe to form a second nanoparticle probe having the first member of the first specific binding pair;
(g) contacting the second nanoparticle probe with one or more third nanoparticle probes to form an aggregate probe in the presence of the second nanoparticle probe, the third nanoparticle probes comprising a nanoparticle having a non-nucleic acid linker molecule bound thereto, wherein a first end of the linker is bound to the third nanoparticle and a second end of the linker is bound to a second member of the first specific binding pair; and
(h) detecting for the presence or absence of the third nanoparticle probe, wherein the presence or absence of the third nanoparticle probe is indicative of the presence or absence of the target analyte in the sample.

58. The method of claim 57, wherein subsequent to step (g) but prior to step (h), further comprising step (g1) isolating the aggregate probe; (g2) releasing the third nanoparticle probe from the aggregate probe; (g3) capturing the third nanoparticle probe onto a second substrate having the first member of the first specific binding pair; and (g4) washing the second substrate to remove any unbound third nanoparticle probe.

59. The method of claim 58, wherein step (h) detecting comprises contacting the washed second substrate with a stain.

60. The method of claim 57, wherein the second nanoparticle probe is a gold nanoparticle probe.

61. The method of claim 58, wherein the second substrate is a waveguide and further comprising subsequent to step (g4), step (g5) illuminating the substrate and observing for any changes in the intensity of light scattered.

62. A nanoparticle probe for detecting for the presence of a target analyte, wherein the target analyte is a first member of a first specific binding pair, the probe comprising a nanoparticle having bound thereto a second member of the first specific binding pair, the second member of the first specific binding pair labeled with a first member of a second specific binding pair.

63. The probe of claim 62, wherein the second specific binding pair comprises is biotin/streptavidin or biotin/avidin.

64. The probe of claim 62, wherein the second member of the first specific binding pair is a target specific antibody and the first member of the second specific binding pair is biotin.

65. A method for detecting for the presence or absence of a target analyte in a sample, wherein the target analyte is a first member of a first specific binding pair, the method comprising:
(a) providing a first substrate;
(b) providing a first nanoparticle probe comprising a nanoparticle having bound thereto a second member of the first specific binding pair, the second member of the first specific binding pair labeled with a first member of a second specific binding pair;
(c) immobilizing the target analyte onto the first substrate;
(d) contacting the immobilized target analyte with the first nanoparticle probe under conditions effective to allow for binding interactions between the target analyte and the first nanoparticle probe to form a complex on the substrate in the presence of the target analyte;
(e) washing the substrate to remove unbound first nanoparticle probes;
(f) releasing the first nanoparticle probe; and
(g) detecting for the presence or absence of the first nanoparticle probe, wherein the presence or absence of the first nanoparticle probe is indicative of the presence or absence of the target analyte in the sample.

66. The method of claim 65, wherein subsequent to step (f) and prior to step (g), further comprising steps (f1) capturing the first nanoparticle probe onto a second substrate having a second member of the first specific binding pair under conditions effective to allow binding interactions between the first nanoparticle probe and the second member of the first specific binding pair to form a complex on the second substrate in the presence of a second nanoparticle probe; and (f2) washing the second substrate to remove any unbound second nanoparticle probe.

67. The method of claim 66, wherein step (g) detecting comprises contacting the washed second substrate with a stain.

68. The method of claim 67, wherein the second nanoparticle probe is a gold nanoparticle probe.

69. The method of claim 66, wherein the second substrate is a waveguide and step (g) comprises illuminating the substrate subsequent to step (f2) and observing for any changes in the intensity of light scattered.

70. A nanoparticle probe for detecting for the presence of a target analyte, the probe comprising a nanoparticle having bound thereto (i) a specific binding complement of a target analyte; and (ii) a first member of a first specific binding pair.

71. The probe of claim 70, wherein the first specific binding pair comprises is biotin/streptavidin or biotin/avidin.

72. The probe of claim 70, wherein the specific binding complement of the target analyte is a target specific antibody and the first member of the first specific binding pair is streptavidin.

73. A method for detecting for the presence or absence of a target analyte in a sample, wherein the target analyte has at least two binding sites, the method comprising:
(a) providing a first substrate;
(b) providing a first nanoparticle probe comprising a nanoparticle having bound thereto (i) a specific binding complement of a target analyte; and (ii) a first member of a first specific binding pair;
(c) immobilizing the target analyte onto the first substrate;
(d) contacting the immobilized target analyte with the first nanoparticle probe under conditions effective to allow for binding interactions between the target analyte and the first nanoparticle probe to form a complex on the substrate in the presence of the target analyte;
(e) washing the substrate to remove unbound first nanoparticle probes;
(f) releasing the first nanoparticle probe; and
(g) detecting for the presence or absence of the first nanoparticle probe, wherein the presence or absence of the first nanoparticle probe is indicative of the presence or absence of the target analyte in the sample.

74. The method of claim 73, wherein subsequent to step (f) and prior to step (g), further comprising steps (f1) capturing the first nanoparticle probe onto a second substrate having a second member of the first specific binding pair under conditions effective to allow binding interactions between the first nanoparticle probe and the second member of the first specific binding pair to form a complex on the second substrate in the presence of the first nanoparticle probe; and (f2) washing the second substrate to remove any unbound first nanoparticle probe.

75. The method of claim 74, wherein step (g) detecting comprises contacting the washed second substrate with a stain.

76. The method of claim 75, wherein the second nanoparticle probe is a gold nanoparticle probe.

77. The method of claim 74, wherein the second substrate is a waveguide and step (g) comprises illuminating the substrate subsequent to step (f2) and observing for any changes in the intensity of light scattered.

78. A method for detecting for the presence or absence of a target analyte in a sample, wherein the target analyte has at least two binding sites, the method comprising:
(a) providing a first substrate and a second substrate;
(b) labeling a sample believed to have the target analyte with a first member of a first specific binding pair;
(c) immobilizing labeled target analyte onto the first substrate;
(d) washing the first substrate to remove unbound labeled target analyte;
(e) releasing the labeled target analyte;
(f) recapturing the labeled target analyte onto the second substrate;
(g) contacting the recaptured target analyte with the a nanoparticle probe comprising a nanoparticle having a second member of the first specific binding pair under conditions effective to allow for binding between the recaptured labeled target analyte and the first nanoparticle probe to form a complex on the substrate in the presence of the labeled target analyte;
(h) washing the substrate to remove unbound nanoparticle probes; and
(i) detecting for the presence or absence of the nanoparticle probe, wherein the presence or absence of the nanoparticle probe is indicative of the presence or absence of the target analyte in the sample.

79. The method of claim 78, wherein step (i) detecting comprises contacting the washed second substrate with a stain.

80. The method of claim 79, wherein the nanoparticle probe is a gold nanoparticle probe.

81. The method of claim 78, wherein the second substrate is a waveguide and step (i) comprises illuminating the substrate subsequent to step (h) and observing for any changes in the intensity of light scattered.

82. A method for detecting for the presence or absence of a target analyte in a sample, wherein the target analyte has at least two binding sites, the method comprising:
(a) providing a first substrate and a second substrate;
(b) providing a first nanoparticle probe comprising a nanoparticle having (i) a first member of a first specific binding pair bound thereto and (ii) a specific binding complement to the target analyte, the specific binding complement labeled with a second member of the first specific binding pair;
(c) providing a second nanoparticle probe comprising a nanoparticle having a non-nucleic acid linker molecule bound thereto, wherein a first end of the linker is bound to the second nanoparticle and a second end of the linker is bound to a second member of the first specific binding pair;
(d) immobilizing the target analyte onto the first substrate;
(e) contacting the immobilized target analyte with the nanoparticle probe under conditions effective to allow for binding interactions between the target analyte and the first nanoparticle probe to form a complex on the substrate in the presence of the target analyte;
(f) washing the first substrate to remove unbound first nanoparticle probes;
(g) releasing the first member of the first specific binding pair from the first nanoparticle probe;
(h) immobilizing the first member of the first specific binding pair onto the second substrate;
(i) washing the second substrate to remove unbound first member of the specific binding pair;
(j) contacting the captured first member of the first specific binding pair on the second substrate with the second nanoparticle probe under conditions effective to allow for binding between the captured first member of the first specific binding pair and the second nanoparticle probe to form a complex in the presence of the first member;
(k) washing the second substrate so as to remove unbound second nanoparticle probes; and
(l) detecting for the presence or absence of the second nanoparticle probe, wherein the presence or absence of the second nanoparticle probe is indicative of the presence or absence of the target analyte in the sample.

83. The method of claim 82, wherein step (l) detecting comprises contacting the washed second substrate with a stain.

84. The method of claim 83, wherein the second nanoparticle probe is a gold nanoparticle probe.

85. The method of claim 82, wherein the second substrate is a waveguide and step (l) comprises illuminating the substrate and observing for any changes in the intensity of light scattered.

86. A method for detecting for the presence or absence of a target analyte in a sample, wherein the target analyte has at least two binding sites, the method comprising:
(a) providing a first substrate;
(b) providing a first particle probe comprising a polyacrylic acid polymer having bound thereto (i) a specific binding complement of the target analyte; and (ii) a first member of a first specific binding pair;
(c) immobilizing the target analyte onto the first substrate;
(d) contacting the immobilized target analyte with the first particle probe under conditions effective to allow for binding interactions between the target analyte and the first particle probe to form a complex on the first substrate in the presence of the target analyte;
(e) washing the first substrate to remove unbound first particle probes;
(f) denaturing the first particle probe to form fragments; and
(g) detecting for the presence or absence of the fragments, wherein the presence or absence of the fragments is indicative of the presence or absence of the target analyte in the sample.

87. The method of claim 86, wherein subsequent to step (f) and prior to step (g), further comprising steps (f1) capturing the fragments onto a second substrate having a second member of the first specific binding pair under conditions effective to allow binding interactions between the fragments and the second member of the first specific binding pair and form a complex on the second substrate in the presence of the fragments; (f2) washing the second substrate to remove any unbound fragments; and (f3) contacting the fragments bound to the second substrate with a nanoparticle probe comprising a nanoparticle having bound thereto the second member of the first specific binding pair.

88. The method of claim 87, wherein step (g) detecting comprises contacting the washed second substrate with a stain.

89. The method of claim 88, wherein the nanoparticle probe is a gold nanoparticle probe.

90. The method of claim 87, wherein the second substrate is a waveguide and step (g) comprises illuminating the substrate subsequent to step (f3) and observing for any changes in the intensity of light scattered.

91. A method for detecting for the presence or absence of a target analyte in a sample, wherein the target analyte has at least two binding sites, the method comprising:
(a) providing a first substrate and a second substrate having a first member of a first specific binding pair bound thereto;
(b) providing a specific binding complement to the target analyte, the specific binding complement labeled with a second member of the first specific binding pair;
(c) providing a nanoparticle probe comprising a nanoparticle having the second member of the first specific binding pair bound thereto;
(d) immobilizing the target analyte onto the first substrate;
(e) contacting the immobilized target analyte with the specific binding complement under conditions effective to allow for binding between the target analyte and the specific binding complement to form a complex on the first substrate in the presence of the target analyte;
(f) washing the first substrate to remove unbound specific binding complement;
(g) releasing specific binding complement;
(h) capturing the released specific binding complement onto the second substrate;
(i) washing the second substrate to remove unbound specific binding complements;
(j) contacting the captured specific binding complement on the second substrate with the nanoparticle probe under conditions effective to allow for binding between the captured specific binding complement and the nanoparticle probe to form a complex in the presence of captured specific binding complement;
(k) washing the second substrate so as to remove unbound nanoparticle probe; and
(l) detecting for the presence or absence of the nanoparticle probe, wherein the presence or absence of the nanoparticle probe is indicative of the presence or absence of the target analyte in the sample.

92. The method of claim 91, wherein step (l) detecting comprises contacting the washed second substrate with a stain.

93. The method of claim 92, wherein the nanoparticle probe is a gold nanoparticle probe.

94. The method of claim 91, wherein the second substrate is a wave guide and step (l) comprises illuminating the substrate and observing for any changes in the intensity of light scattered.

95. A kit for detecting for one or more target analytes in a sample, the kit comprising the nanoparticle probe of any one of claims 1, 40, 54, 62 and 70 and an optional substrate.
